(19) United States
(12) Patent Application Publication
(10) Pub. No.: US 2026/0125631 A1
(43) Pub. Date: May 7, 2026

(54) SIMULATION APPARATUS, SIMULATION SYSTEM, AND SIMULATION METHOD FOR SIMULATING CELL PROLIFERATION BY A CELL CULTURE APPARATUS

(71) Applicant: Terumo Kabushiki Kaisha, Tokyo (JP)

(72) Inventor: Nozomu YAMAZAKI, Kanagawa (JP)

(21) Appl. No.: 19/417,613

(22) Filed: Dec. 12, 2025

Related U.S. Application Data
(63) Continuation of application No. PCT/JP2024/021739, filed on Jun. 14, 2024.

(30) Foreign Application Priority Data
Jun. 16, 2023 (JP) 2023-098863

(51) Int. Cl. C12M 1/34; C12M 1/00

(52) U.S. Cl. CPC C12M 41/32; C12M 29/16; C12M 29/18

(57) ABSTRACT

A simulation apparatus configured to simulate a change in concentration of protein in an inner circulation path using a supply speed, an exchange speed, and a deterioration speed, a predetermined or selected culture condition being applied in the circulation path, the supply speed being a first speed at which a first concentration of the protein in the circulation path changes as a result of the supply of the culture medium to the circulation path, the exchange speed being a second speed at which a second concentration of the protein in the circulation path changes as a result of inflow and outflow of the culture medium between the circulation path and an outer region of the circulation path, and the deterioration speed being a third speed at which a third concentration of the protein in the circulation path changes as a result of deterioration of the protein.

### CROSS-REFERENCE TO RELATED APPLICATIONS

The present application is a continuation of the International Patent Application No. PCT/JP2024/021739 filed Jun. 14, 2024, which designated the U.S. and claims the benefit of priority from Japanese Patent Application No. JP2023098863 filed Jun. 16, 2023. The entire disclosures of the above-identified applications are incorporated herein by reference.

### FIELD

The present invention relates to a simulation apparatus, a simulation system, and a simulation method for simulating cell proliferation by a cell culture apparatus.

### BACKGROUND

JP 2020-171241 A discloses a cell culture apparatus. including a bioreactor, a supply unit, a recovery unit, and a plurality of flow paths. Some flow paths of the cell culture apparatus form a circulation path with the bioreactor. The supply unit is configured to supply a cell-containing solution and a culture medium (culture solution) to the bioreactor, which cultures cells. During cell culture, a part of the culture medium (e.g., first culture medium) circulates in the circulation path and a part of the culture medium (e.g., second culture medium) is discharged as a waste liquid. The recovery unit is configured to recover the cultured cells.

Supply of nutrients (e.g., glucose, glutamine, various amino acids, the like, or any combination thereof), supply of gases (e.g., oxygen, carbon dioxide, the like, or any combination thereof), and discharge of waste products (e.g., lactic acid, ammonia, the like, or any combination thereof) are important for cell culture. Protein supply is also important for cell culture. Therefore, bovine serum (which includes albumin and growth factors), growth factors, cytokines, the like, or any combination thereof are added to the culture medium.

When protein supplied to the cell is insufficient, the cell does not proliferate. In contrast, when the amount of protein to be supplied to the cell is too large, the cell proliferation may be inhibited. Furthermore, a unit price of some proteins (e.g., growth factors, cytokines, and the like) is often high. Accordingly, it is desirable to appropriately control the amount of protein to be supplied to cells.

### SUMMARY

At least one example embodiment relates to a simulation apparatus that is configured to execute a simulation of cell proliferation in a cell culture apparatus, where the cell culture apparatus is configured to supply a culture medium (e.g., containing protein) to a circulation path that is defined by an inner pore of a cylindrical hollow fiber membrane (i.e., an inner circulation path) such that cells are cultured in the inner pore. The simulation apparatus may include a simulation execution unit that is configured to simulate a change in concentration of the protein in the inner circulation path while cell proliferation occurs under a predetermined or selected culture condition at least using a supply speed which is a speed at which the concentration of the protein changes in the inner circulation path as a result of a culture medium being supplied to the inner circulation path, an exchange speed which is a speed at which the concentration of the protein changes in the inner circulation path as a result inflow and outflow of the culture medium between an inside of the inner pore and an outside of the inner pore via the hollow fiber membrane, and a deterioration speed which is a speed at which the concentration of the protein changes in the inner circulation path due to deterioration of the protein.

From a relationship between a molecular weight cutoff of the hollow fiber membrane and a size of protein, a behavior of the protein diffusing into and out of the cylindrical hollow fiber membrane may differ for each type of protein. The simulation may be executed using not only the supply speed and the deterioration speed but also the exchange speed which improves the accuracy of the simulation of the change in concentration of protein. The user may appropriately control (i.e., adjust) an amount of protein by reflecting the simulation result in the real culture.

In at least one example embodiment, the simulation may further include a display control unit that is configured to control a display unit of the cell culture apparatus, including, for example, to display information according to the concentration change obtained by the simulation.

In at least one example embodiment, the simulation of the change in concentration of the protein in the inner circulation path may further include using a consumption speed which is a speed at which the concentration of the protein changes in the inner circulation path as a result of the consumption of the protein by the cells.

In at least one example embodiment, where a part of the culture medium is discarded via the inner circulation path during cell culture, the simulation of the change in concentration of the protein in the inner circulation path may further include using a discard speed which is a speed at which the concentration of the protein changes in the inner circulation path as a result of discarding a part of the culture medium via the inner circulation path.

In at least one example embodiment, the simulation of the change in concentration of the protein in the inner circulation path may further using an adsorption rate which is a ratio of the protein that cannot be consumed by the cells as the protein is adsorbed, aggregated, and deposited on at least one of an inside of the inner circulation path and an inside of an outer circulation path. The outer circulation path is a circulation path including a region between the hollow fiber membrane and a housing that stores the hollow fiber membrane.

In at least one example embodiment, the cell culture apparatus may be configured to supply a culture medium that does not include the protein to the outer circulation path.

In at least one example embodiment, for example, when culturing adherent cells, the cell culture apparatus may be configured to discard the culture medium via a waste liquid flow path that is connected to the inner circulation path.

In at least one example embodiment, for example, when culturing floating cells, the cell culture apparatus may be configured to discard the culture medium via a waste liquid flow path that is connected to the outer circulation path.

In at least one example embodiment, the cell culture apparatus may include an inside of the inner circulation path and the outer circulation path, when a part of the culture medium is discarded via the outer circulation path during cell culture, the simulation execution unit may be configured to execute the simulation of the change in concentration of the protein in the outer circulation path along with the cell proliferation under the culture condition by using at least a discard speed which is a speed at which the concentration of the protein changes in the outer circulation path by discarding a part of the culture medium via the outer circulation path, the exchange speed, and the deterioration speed.

At least one example embodiment t relates to a simulation system that is configured to execute a simulation of cell proliferation in a cell culture apparatus, where the cell culture apparatus is configured to supply a culture medium (e.g., containing protein) to a circulation path that is defined by an inner pore of a cylindrical hollow fiber membrane (i.e., an inner circulation path) such that the cells are cultured in the inner pore. The simulation system may include a simulation execution unit that is configured to simulate a change in concentration of the protein in the inner circulation path while cell proliferation occurs under a predetermined or selected culture condition using a supply speed which is a speed at which the concentration of the protein changes in the inner circulation path as a result of a culture medium being supplied to the inner circulation path, an exchange speed which is a speed at which the concentration of the protein changes in the inner circulation path as a result of inflow and outflow of the culture medium between an inside of the inner pore and an outside of the inner pore via the hollow fiber membrane, and a deterioration speed which is a speed at which the concentration of the protein changes in the inner circulation path due to deterioration of the protein. The simulations system may also include a display control unit that is configured to control a display unit of the cell culture apparatus, including, for example, to display information according to the change in concentration obtained by the simulation.

In at least one example embodiment, the simulation may be executed using not only the supply speed and the deterioration speed but also the exchange speed, which improves the accuracy of the simulation of the change in concentration of protein. The user may appropriately control (i.e., adjust) an amount of protein by reflecting the simulation result in the real culture.

At least one example embodiment relates to a simulation method that includes executing a simulation of cell proliferation in a cell culture apparatus, where the cell culture apparatus is configured to supply a culture medium (e.g., containing protein) to a circulation path that is defined by an inner pore of a cylindrical hollow fiber membrane (i.e., an inner circulation path) such that the cells are cultured in the inner pore. The simulation method may include a simulation step that includes simulating a change in concentration of the protein in the inner circulation path while cell proliferation occurs under a predetermined or selected culture condition using a supply speed which is a speed at which the concentration of the protein changes in the inner circulation path as a result of a culture medium being supplied to the inner circulation path, an exchange speed which is a speed at which the concentration of the protein changes in the inner circulation path as a result of inflow and outflow of the culture medium between an inside of the inner pore and an outside of the inner pore via the hollow fiber membrane, and a deterioration speed which is a speed at which the concentration of the protein changes in the inner circulation path due to deterioration of the protein.

In at least one example embodiment, the simulating may be occur using not only the supply speed and the deterioration speed but also the exchange speed, which improves the accuracy of the simulation of the change in concentration of protein. The user may appropriately control (i.e., adjust) an amount of protein by reflecting the simulation result in the real culture.

In at least one example embodiment, the simulation method may further include a display step that includes allowing a display unit of the cell culture apparatus to display information according to the change in concentration obtained by the simulation.

In at least one example embodiment, the simulating of the change in concentration of the protein in the inner circulation path may further include using a consumption speed which is a speed at which the concentration of the protein changes in the inner circulation path by consumption of the protein by the cells.

In at least one example embodiment, for example, when a part of the culture medium is discarded via the inner circulation path during cell culture, the simulating of the change in concentration of the protein in the inner circulation path may further include using a discard speed which is a speed at which the concentration of the protein changes in the inner circulation path by discarding a part of the culture medium via the inner circulation path.

In at least one example embodiment, the simulating of the change in concentration of the protein in the inner circulation path may further include using an adsorption rate which is a ratio of the protein that cannot be consumed by the cells as the protein is adsorbed, aggregated, and deposited on at least one of an inside of the inner circulation path and an inside of the outer circulation path.

In at least one example embodiment, the cell culture apparatus may be configured to supply a culture medium that does not include the protein to the outer circulation path.

In at least one example embodiment, for example, when culturing adherent cells, the cell culture apparatus may be configured to discard the culture medium via a waste liquid flow path connected to the inner circulation path.

In at least one example embodiment, for example, when culturing floating cells, the cell culture apparatus may be configured to discard the culture medium via a waste liquid flow path connected to the outer circulation path.

In at least one example embodiment, the cell culture apparatus may include an inside of the inner circulation path and the outer circulation path, when a part of the culture medium is discarded via the outer circulation path during cell culture, the simulating of the change in concentration of the protein in the outer circulation path along with the cell proliferation under the culture condition may be executed using at least a discard speed which is a speed at which the concentration of the protein changes in the outer circulation path by discarding a part of the culture medium via the outer circulation path, the exchange speed, and the deterioration speed.

According to various aspects, using simulation apparatus and/or the simulation system and/or the simulation method the amount of protein can be appropriately controlled.

### DRAWINGS

FIG. 1 is a diagram illustrating a configuration of a cell culture system in accordance with at least one example embodiment.

FIG. 2 is a diagram illustrating a configuration of a control unit of the cell culture apparatus of FIG. 1 in accordance with at least one example embodiment.

FIG. 3 is a diagram illustrating a configuration of a simulation apparatus for use with the cell culture system of FIG. 1 in accordance with at least one example embodiment.

FIG. 4 is a table illustrating parameter information in accordance with at least one example embodiment.

FIG. 5 is a diagram illustrating an input screen displayed on a display unit of the cell culture system of FIG. 1 in accordance with at least one example embodiment.

FIG. 6 is a diagram illustrating a proliferation data screen displayed on the display unit of the cell culture system of FIG. 1 in accordance with at least one example embodiment.

FIG. 7 is a diagram illustrating a feedback condition screen displayed on the display unit of the cell culture system of FIG. 1 in accordance with at least one example embodiment.

FIG. 8 is a diagram illustrating a results screen displayed on the display unit of the cell culture system of FIG. 1 in accordance with at least one example embodiment.

FIG. 9 is a diagram illustrating a result screen displayed on the display unit of the cell culture system of FIG. 1 in accordance with at least one example embodiment.

FIG. 10 is a flowchart illustrating a flow of a cell culture method performed using the cell culture system of FIG. 1 in accordance with at least one example embodiment.

FIG. 11 is a diagram illustrating an operation state of the cell culture apparatus of FIG. 1 at the time of cell culture in a first culture form in accordance with at least one example embodiment.

FIG. 12 is a diagram illustrating an operation state of the cell culture apparatus of FIG. 1 at the time of cell collection in the first culture form in accordance with at least one example embodiment.

FIG. 13 is a diagram illustrating an operation state of the cell culture apparatus of FIG. 1 at the time of cell culture in a second culture form in accordance with at least one example embodiment.

FIG. 14 is a diagram illustrating an operation state of the cell culture apparatus of FIG. 1 at the time of cell culture in a third culture form in accordance with at least one example embodiment.

FIG. 15 is a diagram illustrating an operation state of the cell culture apparatus of FIG. 1 at the time of cell culture in a fourth culture form in accordance with at least one example embodiment.

FIG. 16 is a flowchart illustrating a flow of the cell culture performed using the cell culture apparatus of FIG. 1 in accordance with at least one example embodiment.

FIG. 17 is a diagram illustrating an operation state of the cell culture apparatus of FIG. 1 at the time of cell culture in accordance with at least one example embodiment.

FIG. 18 is a diagram illustrating an operation state of the cell culture apparatus of FIG. 1 at the time of cell detachment in accordance with at least one example embodiment.

FIG. 19 is a diagram illustrating an operation state of the cell culture apparatus of FIG. 1 at the time of cell recovery in accordance with at least one example embodiment.

FIG. 20 is a diagram illustrating a configuration of a simulation system for use with the cell culture system of FIG. 1 in accordance with at least one example embodiment.

FIG. 21 is a table illustrating a specific example of numerical values of parameter information in accordance with at least one example embodiment.

FIG. 22 is a table illustrating a first culture condition in accordance with at least one example embodiment.

FIG. 23 is a table illustrating a second culture condition in accordance with at least one example embodiment.

FIG. 24 is a table illustrating a third culture condition in accordance with at least one example embodiment.

FIG. 25 is a graphical illustration demonstrating transition of a concentration of protein in a first circulation flow path in accordance with at least one example embodiment.

FIG. 26 is a graphical illustration demonstrating transition of the concentration of protein in the first circulation flow path in accordance with at least one example embodiment.

FIG. 27 is a graphical illustration demonstrating transition of the concentration of protein in the first circulation flow path in accordance with at least one example embodiment.

FIG. 28 is a table illustrating a fourth culture condition in accordance with at least one example embodiment.

FIG. 29 is a graphical illustration demonstrating transition of the concentration of protein in the first circulation flow path in accordance with at least one example embodiment.

### DETAILED DESCRIPTION

In cell culture, when using a hollow fiber membrane, because of a relationship between a fractionated size of the hollow fiber membrane and a molecular weight of a protein, a protein smaller than the fractionated size flows out of the hollow fiber membrane during a culture period. Accordingly, it is often difficult to accurately determine a protein concentration.

Examples of cells to be cultured include adherent cells that grow using the hollow fiber membrane as a scaffold and floating cells that float and grow within the hollow fiber membrane. In order to culture cells, a cell culture form according to a cell type is required. In order to accurately determine the protein concentration, a method for predicting a change in protein concentration according to the cell culture form is required.

Further, it is often necessary to change various cell culture forms according to a cell proliferation state, including, for example, to maintain a nutrient source in a cell proliferation period in a culture initial stage and enrich the nutrient source in culture middle to later stages. It is often difficult to predict the change in protein concentration during a series of culture periods.

The simulation apparatus and/or the simulation system and/or the simulation method detailed below allows the protein concentration to be accurately determined. The simulation apparatus and/or the simulation system and/or the simulation method detailed below allows change in protein concentration to be predicted according to the cell culture form. The simulation apparatus and/or the simulation system and/or the simulation method detailed below allow the change in protein concentration in a series of culture periods to be predicted.

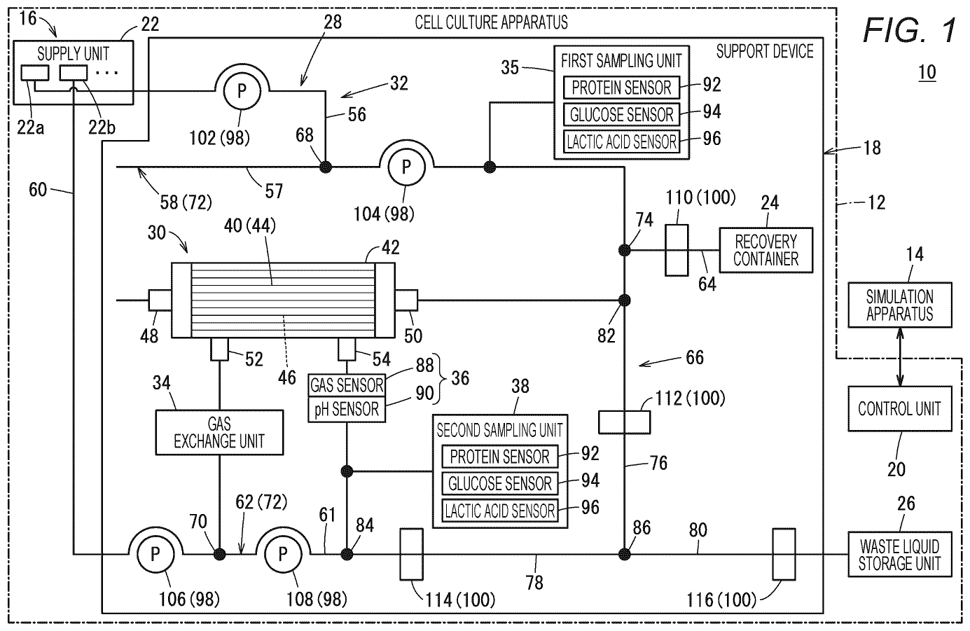

FIG. 1 is a diagram illustrating a configuration of a cell culture system 10. The cell culture system 10 cultures (i.e., proliferates) cells separated from biological tissue in a culture medium. The cells used in the cell culture system 10 may include mammalian cells or cells derived from a mammal. The cells used in the cell culture system 10 may include adherent cells, floating cells, or a combination of adherent cells and floating cells. Examples of the adherent cells include human embryonic kidney cells (HEK293 cells), embryonic stem cells (ES cells), induced pluripotent stem cells (iPS cells), mesenchymal stem cells, fibroblast cells, endothelial cells, neural stem cells, the like, or any combination thereof. Examples of the floating cells include Jurkat cells (human cellular leukemia-derived cells), T cells, regulatory T cells, tumor-infiltrating lymphocytes, CAR-T cells, CD34 positive cells, the like, or any combination thereof.

The cell culture system 10 may include a cell culture apparatus 12 and a simulation apparatus 14. The cell culture apparatus 12 may include a cell culture circuit 16, a support device 18, and a control unit 20. A liquid may flow in the cell culture circuit 16. The liquid may include a cell fluid, a culture medium, a cleaning liquid, a detaching liquid, or any combination thereof. The cell fluid is a solution containing cells. The culture medium is a culture solution for proliferating the cells. The culture medium may be selected according to the cells to be cultured. The culture media may include a basal medium, a complete medium, or a combination of basal medium and complete medium. The basal medium may include, for example, a minimum essential medium (MEM). The complete medium may include the basal medium and a protein. The protein may include albumin, growth factors, cytokines, the like, or any combination thereof. For example, bovine serum that includes albumin, growth factors, the like, or any combination thereof may be is added to the basal medium to form the complete medium. The cleaning liquid may be selected to clean the inside of the cell culture circuit 16. The cleaning liquid may include, for example, water, a buffer solution, physiological saline, the like, or any combination thereof. Examples of the buffer solution include phosphate buffered saline (PBS), tris-buffered saline (TBS), the like, or any combination thereof. The detaching liquid is selected to detach cells from a bioreactor 30 of the cell culture circuit 16. The detaching liquid may include, for example, trypsin, an EDTA solution, the like, or any combination thereof.

The cell culture circuit 16 is a single-use disposable and discarded after every use. That is, the cell culture circuit 16 is discarded every time a predetermined or selected number of cells is cultured. The cell culture circuit 16 may include a supply unit 22, a recovery container 24, a waste liquid storage unit 26, and a culture main body 28.

The supply unit 22 may be configured to supply the liquid (such as the cell fluid, the culture medium, the cleaning liquid, the detaching liquid, or a combination thereof) to the culture main body 28. The supply unit 22 includes a first supply unit 22a and a second supply unit 22b. The recovery container 24 is configured to recover the cells cultured in the culture main body 28. The waste liquid storage unit 26 is configured to store a waste liquid generated in the culture main body 28. In at least one example embodiment, at least one of the recovery container 24 and the waste liquid storage unit 26 may be defined by a medical bag that is obtained by shaping a soft resin material into a bag shape. In at least one example embodiment, at least one of the recovery container 24 and the waste liquid storage unit 26 may be defined by a tank or other container formed of a hard material.

The culture main body 28 may include the bioreactor 30, a flow path 32, a gas exchange unit 34, a first sampling unit 35, a sensor unit 36, and a second sampling unit 38.

The bioreactor 30 may include a plurality of hollow fiber membranes 40 and a cylindrical housing 42. The plurality of hollow fiber membranes 40 may be stored in the housing 42. In at least on example embodiment, the hollow fiber membrane 40 may have a cylindrical shape. The hollow fiber membrane 40 may include a pore (i.e., inner pore) that penetrates from a first end to a second end of the hollow fiber membrane 40. The first end of each hollow fiber membrane 40 may be fixed to a first end of the housing 42. The second end of each hollow fiber membrane 40 may be fixed to a second end of the housing 42. The hollow fiber membrane 40 may be formed of a polymer material.

The bioreactor 30 may include a first region 44 and a second region 46. The first region 44 may be the inner pore of the plurality of hollow fiber membranes 40. The first region 44 may be a region where cells are present in the culture medium. The first region 44 may also be referred to as a culture region. The second region 46 may be a space between an inner peripheral surface of the housing 42 and outer peripheral surfaces of the plurality of hollow fiber membranes 40. The second region 46 may be a non-culture region of an inner region of the bioreactor 30. The hollow fiber membrane 40 may include a plurality of pores (not illustrated). The first region 44 and the second region 46 may communicate with each other via the plurality of pores. A diameter of each pore is a size that allows passage of low molecules (e.g., water, ions, oxygen, lactate, the like, or any combination thereof) while blocking passage of macromolecules (e.g., cells and the like). The diameter of each pore may be set to, for example, greater than or equal to about 0.005 micrometers to less than or equal to about 10 micrometers.

A first inlet port 48, a first outlet port 50, a second inlet port 52, and a second outlet port 54 may be attached to the housing 42. The first inlet port 48 may be attached to the first end of the housing 42. The first inlet port 48 may communicate with the first region 44 via an inlet located at the first end of the plurality of hollow fiber membranes 40. The first outlet port 50 may be attached to the second end of the housing 42. The first outlet port 50 may communicate with the first region 44 via an outlet located at the second end of the plurality of hollow fiber membranes 40.

The second inlet port 52 and the second outlet port 54 may be attached to an outer peripheral surface of the housing 42. The second inlet port 52 may be located between the center of the housing 42 and the first inlet port 48 in a longitudinal direction of the housing 42. The second outlet port 54 may be located between the center of the housing 42 and the first outlet port 50 in the longitudinal direction of the housing 42. The second inlet port 52 and the second outlet port 54 may both communicate with the second region 46.

The flow path 32 may include a plurality of tubes through which a liquid flows. Each tube may be formed of a soft resin material. The flow path 32 may include a first supply flow path 56, a first communication flow path 57, a second supply flow path 60, a second communication flow path 61, a recovery flow path 64, and a waste liquid flow path 66.

One end of the first supply flow path 56 may be connected to the first supply unit 22a. The first supply unit 22a may be configured to supply the cell fluid, the complete medium, the basal medium, the cleaning liquid, and the detaching liquid one by one to the first supply flow path 56 at one or more predetermined or selected times. The other end of the first supply flow path 56 may be connected to a first junction 68 of the first communication flow path 57.

The first junction 68 may be located between a first end and a second end of the first communication flow path 57. The first end of the first communication flow path may be connected to the first inlet port 48. That is, the first communication flow path 57 may communicate with the first region 44 in the inner pore of each hollow fiber membrane 40 via the first inlet port 48. The second end of the first communication flow path 57 may be connected to the first outlet port 50. That is, the first communication flow path 57 may communicate with the first region 44 in the inner pore of each hollow fiber membrane 40 via the first outlet port 50. A closed circuit formed by the first communication flow path 57 and the first region 44 may be referred to as a first circulation flow path (i.e., inner circulation path) 58.

One end of the second supply flow path 60 may be connected to the second supply unit 22b. The second supply unit 22b may be configured to supply the basal medium and the cleaning liquid one by one to the second supply flow path 60 at a predetermined or selected times. The other end of the second supply flow path 60 may be connected to a second junction 70 of the second communication flow path 61.

The second junction 70 may be located between a first end and a second end of the second communication flow path 61. The first end of the second communication flow path 61 may be connected to the second inlet port 52. That is, the second communication flow path 61 may communicate with the second region 46 outside the inner pore of each hollow fiber membrane 40 via the second inlet port 52. The second end of the second communication flow path 61 may be connected to the second outlet port 54. That is, the second communication flow path 61 may communicate with the second region 46 outside the inner pore of each hollow fiber membrane 40 via the second outlet port 54. A closed circuit formed by the second communication flow path 61 and the second region 46 may be referred to as a second circulation flow path (i.e., outer circulation path) 62. Hereinafter, the first circulation flow path 58 and the second circulation flow path 62 may be collectively referred to as a “circulation flow path 72”.

The recovery flow path 64 may extend from the first circulation flow path 58. One end of the recovery flow path 64 may be connected to a recovery branch 74 of the first circulation flow path 58. The recovery branch 74 may be located between the first junction 68 and the first outlet port 50 in the first circulation flow path 58. The other end of the recovery flow path 64 may be connected to the recovery container 24.

A liquid to be discarded from the circulation flow path 72 may flow through the waste liquid flow path 66. The waste liquid flow path 66 may include a first waste liquid flow path 76, a second waste liquid flow path 78, and a third waste liquid flow path 80. The first waste liquid flow path 76 may extend from the first circulation flow path 58. One end of the first waste liquid flow path 76 may be connected to the first branch 82 of the first circulation flow path 58. The first branch 82 may be located between the first outlet port 50 and the recovery branch 74 of the first circulation flow path 58. The second waste liquid flow path 78 may extend from the second circulation flow path 62. One end of the second waste liquid flow path 78 may be connected to the second branch 84 of the second circulation flow path 62. The second branch 84 may be located between the second junction 70 and the second outlet port 54 of the second circulation flow path 62. The other end of the first waste liquid flow path 76 and the other end of the second waste liquid flow path 78 may be connected to each other at an intermediate junction 86. One end of the third waste liquid flow path 80 may be connected to the first waste liquid flow path 76 and the second waste liquid flow path 78 at the intermediate junction 86. The other end of the third waste liquid flow path 80 may be connected to the waste liquid storage unit 26.

The gas exchange unit 34 may be attached between the second junction 70 and the second inlet port 52 of the second circulation flow path 62. The gas exchange unit 34 may be configured to allow a gas of a predetermined component to pass through a liquid (basal medium) flowing through the second circulation flow path 62. The gas used in the gas exchange unit 34 may include, for example, components similar to those of air. In other words, the gas may include nitrogen, oxygen, and carbon dioxide. Specifically, the gas may include 75% nitrogen, 20% oxygen, and 5% carbon dioxide in volume ratio.

The first sampling unit 35 may be connected to the first circulation flow path 58. The first sampling unit 35 may be configured to extract a part of a liquid (complete medium) that flows through the first circulation flow path 58 and to measure components contained in the liquid. For example, the first sampling unit 35 may aseptically collect a tube fragment that contains an internal liquid from a tube having a sufficient length using a sterile joining device. The first sampling unit 35 may include a protein sensor 92, a glucose sensor 94, a lactic acid sensor 96, or any combination thereof.

The sensor unit 36 may be attached between the second outlet port 54 and the second branch 84 of the second circulation flow path 62. The sensor unit 36 may include a gas sensor 88 and a pH sensor 90. The gas sensor 88 may be configured to measure a gas concentration of a liquid flowing through the second circulation flow path 62. For example, the gas sensor 88 may include an oxygen sensor and a carbon dioxide sensor. The oxygen sensor may be configured to measure an oxygen concentration of the liquid flowing through the second circulation flow path 62. The carbon dioxide sensor may be configured to measure a carbon dioxide concentration of the liquid flowing through the second circulation flow path 62. The pH sensor 90 may be configured to measure pH (hydrogen ion index) of the liquid flowing through the second circulation flow path 62. The gas sensor 88 and the pH sensor 90 may output measurement results to the control unit 20.

The second sampling unit 38 may be connected to a portion between the second outlet port 54 and the second branch 84 of the second circulation flow path 62. The second sampling unit 38 may be configured to extract a part of the liquid flowing through the second circulation flow path 62 and to measure a component contained in the liquid. The second sampling unit 38 may include a protein sensor 92, a glucose sensor 94, a lactic acid sensor 96, or any combination thereof.

The protein sensor 92 may be configured to measure a protein concentration of the liquid extracted from the second circulation flow path 62. The glucose sensor 94 may be configured to measure a glucose concentration of the liquid extracted from the second circulation flow path 62. The lactic acid sensor 96 may be configured to measure a lactic acid concentration of the liquid extracted from the second circulation flow path 62. The protein sensor 92, the glucose sensor 94, and the lactic acid sensor 96 may output measurements to the control unit 20.

The cell culture circuit 16 may be set in the support device 18. The support device 18 may include a cassette that supports the cell culture circuit 16. The support device 18 may be a reusable product that can be used a number times.

The support device 18 may include a plurality of pumps 98 and a plurality of clamps 100. Each pump of the plurality of pumps 98 may be configured to apply a flow force to the liquid in the flow path 32, for example, by squeezing a wall of the flow path 32. Each pump of the plurality of pumps 98 may include a pressing member (not illustrated). The pressing member may include, for example, a rotating member and a plurality of pressing rollers. The plurality of pressing rollers may be attached to an outer peripheral portion of the rotating member. The plurality of pressing rollers may be arranged at intervals in a circumferential direction of the rotating member. Each pressing roller may rub an outer surface of the wall of the flow path 32.

The plurality of pumps 98 may include a first supply pump 102, a first circulation pump 104, a second supply pump 106, and a second circulation pump 108. A state in which the cell culture circuit 16 is set in the support device 18 is simply referred to as a “set state”.

In the set state, a part of the first supply flow path 56 may be attached to the first supply pump 102. The first supply pump 102 may be configured to apply a flow force in a direction from the supply unit 22 toward the first circulation flow path 58 to the liquid in the first supply flow path 56.

In the set state, a part of the first circulation flow path 58 may be attached to the first circulation pump 104. The first circulation pump 104 may be configured to apply a flow force in a direction from the first outlet port 50 toward the first inlet port 48 to the liquid in the first circulation flow path 58. The first circulation pump 104 may also be configured to apply a flow force in a direction from the first inlet port 48 toward the first outlet port 50 to the liquid in the first circulation flow path 58.

In the set state, a part of the second supply flow path 60 may be attached to the second supply pump 106. The second supply pump 106 may be configured to apply a flow force in a direction from the supply unit 22 toward the second circulation flow path 62 to the liquid in the second supply flow path 60.

In the set state, a part of the second circulation flow path 62 may be attached to the second circulation pump 108. The second circulation pump 108 may be configured to apply a flow force in a direction from the second outlet port 54 toward the second inlet port 52 to the liquid in the second circulation flow path 62. The second circulation pump 108 also be configured to apply a flow force in a direction from the second inlet port 52 toward the second outlet port 54 to the liquid in the second circulation flow path 62.

The plurality of clamps 100 may be configured to close the flow path 32 by pressing the outer surface of the flow path 32 toward an inner surface. For example, the plurality of clamps 100 may include open/close valves. The plurality of clamps 100 may include a recovery clamp 110, a first waste liquid clamp 112, a second waste liquid clamp 114, and a third waste liquid clamp 116.

In the set state, a part of the recovery flow path 64 may be attached to the recovery clamp 110. The recovery clamp 110 may be configured to open and close the recovery flow path 64. In the set state, a part of the first waste liquid flow path 76 may be attached to the first waste liquid clamp 112. The first waste liquid clamp 112 may be configured to open and close the first waste liquid flow path 76. In the set state, a part of the second waste liquid flow path 78 may be attached to the second waste liquid clamp 114. The second waste liquid clamp 114 may be configured to open and close the second waste liquid flow path 78. In the set state, a part of the third waste liquid flow path 80 may be attached to the third waste liquid clamp 116. The third waste liquid clamp 116 may be configured to open and close the third waste liquid flow path 80.

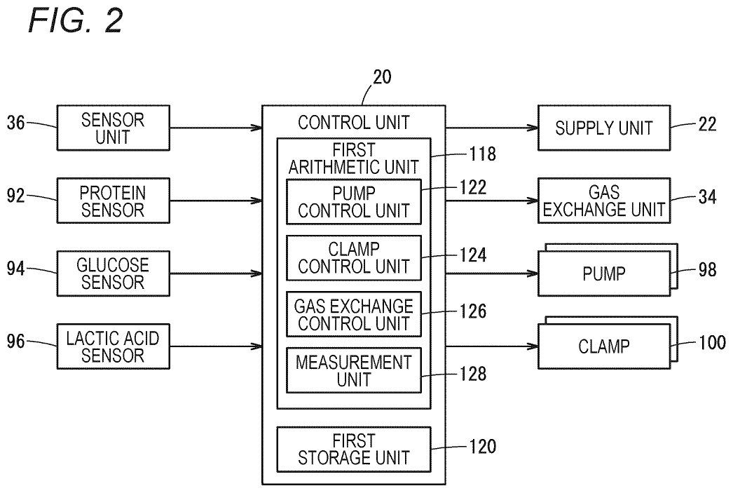

FIG. 2 is a diagram illustrating a configuration of the control unit 20 of the cell culture apparatus 12. The control unit 20 may include a first arithmetic unit 118, a first storage unit 120, and various drive circuits (not illustrated).

The first arithmetic unit 118 may include a processing circuit. In at least one example embodiment, the processing circuit may include a processor, such as a CPU. In at least one example embodiment, the processing circuit may include an integrated circuit, such as an ASIC and an FPGA. The processor may be configured to execute various types of processing by executing one or more programs stored in the first storage unit 120. The control unit 20 may be configured to function as a pump control unit 122, a clamp control unit 124, a gas exchange control unit 126, a measurement unit 128, or any combination thereof. At least a part of the plurality of pieces of processing may be executed by an electronic circuit including a discrete device.

The pump control unit 122 may be configured to control each of the plurality of pumps 98. For example, the pump control unit 122 may be configured to output a command signal to a pump drive circuit (not illustrated). The pump drive circuit may be configured to supply power according to the command signal of the pump control unit 122 to each of the plurality of pumps 98. The clamp control unit 124 may be configured to control each of the plurality of clamps 100. For example, the clamp control unit 124 may be configured to output a command signal to a clamp drive circuit (not illustrated). The clamp drive circuit may be configured to supply power according to the command signal of the clamp control unit 124 to each of the plurality of clamps 100. The gas exchange control unit 126 may be configured to control the gas exchange unit 34. For example, the gas exchange control unit 126 may be configured to output a command signal to a gas exchanger drive circuit (not illustrated). The gas exchanger drive circuit may be configured to supply power according to the command signal of the gas exchange control unit 126 to the gas exchange unit 34. The measurement unit 128 may be configured to acquire the measurement result from the gas sensor 88, the pH sensor 90, the protein sensor 92, the glucose sensor 94, the lactic acid sensor 96, or any combination thereof. The measurement unit 128 may be configured to store the acquired measurement result in the first storage unit 120.

The first storage unit 120 may include a volatile memory and a nonvolatile memory. Examples of the volatile memory may include a RAM and the like. The volatile memory may be used as a working memory of the processor. The volatile memory may be configured to temporarily stores data and the like necessary for processing or an arithmetic operation. Examples of the nonvolatile memory may include a ROM, a flash memory, the like, or any combination thereof. The nonvolatile memory may be used as a memory for storage. The nonvolatile memory may be configured to store a program, a table, a map, the like, or any combination thereof. At least a part of the first storage unit 120 may be provided in the processor, the integrated circuit, and the like described above.

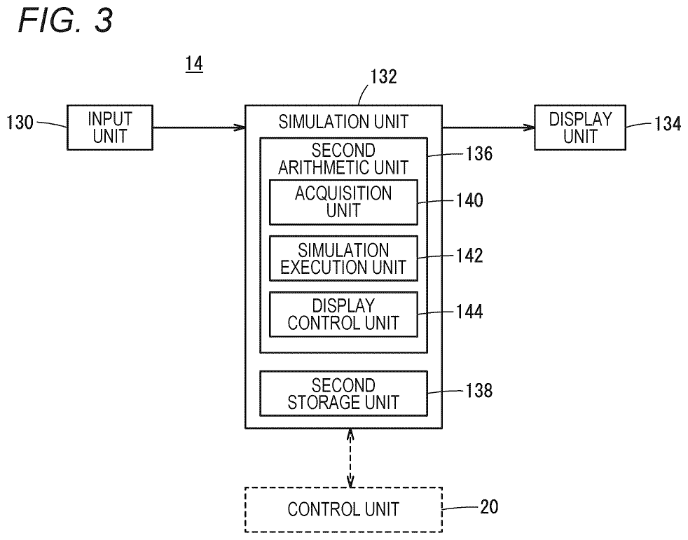

FIG. 3 is a diagram illustrating a configuration of the simulation apparatus 14. The simulation apparatus 14 may include an input unit 130, a simulation unit 132, and a display unit 134. As the simulation apparatus 14, a personal computer, a smartphone, a tablet, the like, or any combination thereof may be used.

The input unit 130 may include a human-machine interface, such as a keyboard, a mouse, and a touch pad. In at least one example embodiment, the input unit 130 may include a human-machine interface integrated with the display unit 134, such as a touch panel. The input unit 130 may be configured to input data according to an operation performed by the user to the simulation unit 132.

The simulation unit 132 may include a second arithmetic unit 136 and a second storage unit 138. The first arithmetic unit 118 and the first storage unit 120 may be used as the second arithmetic unit 136 and the second storage unit 138. That is, in at least one example embodiment, the control unit 20 of the cell culture apparatus 12 may be used as the simulation unit 132. The second arithmetic unit 136 may include a processing circuit. In at least one example embodiment, the processing circuit may include a processor, such as a CPU. In at least one example embodiment, the processing circuit may include an integrated circuit, such as an ASIC and an FPGA. The processor may be configured to execute various types of processing, for example, by executing one or more programs stored in the second storage unit 138. The simulation unit 132 may function as an acquisition unit 140, a simulation execution unit 142, a display control unit 144, or any combination thereof. At least a part of the plurality of pieces of processing may be executed by an electronic circuit including a discrete device.

The acquisition unit 140 may be configured to acquire data from the outside of the second arithmetic unit 136. For example, the acquisition unit 140 may be configured to acquire the data from the input unit 130, the second storage unit 138, the like, or any combination thereof. The acquisition unit 140 may be configured to acquire the data designated by the input unit 130 from the second storage unit 138. The acquisition unit 140 may be configured to acquire the data designated by the input unit 130 from a device (such as the control unit 20) designated by the input unit 130. Using the data acquired by the acquisition unit 140, the simulation execution unit 142 may be configured to execute a simulation of the cell proliferation and change in concentration of protein by the cell culture apparatus 12. The display control unit 144 may be configured to allow the display unit 134 to display various screens. For example, the display control unit 144 may be configured to allow the display unit 134 to display the data stored in the second storage unit 138. The display control unit 144 may be configured to allow the display unit 134 to display information according to a result of the simulation executed by the simulation execution unit 142.

The second storage unit 138 may include a volatile memory and a nonvolatile memory. Examples of the volatile memory may include a RAM and the like. The volatile memory may be used as a working memory of the processor. The volatile memory may be configured to temporarily stores data and the like necessary for processing or an arithmetic operation. Examples of the nonvolatile memory may include a ROM, a flash memory, the like, or any combination thereof. The nonvolatile memory may be used as a memory for storage. The nonvolatile memory may be configured to store a program, a table, a map, the like, or any combination thereof. The nonvolatile memory may be configured to store a simulation program executed by the simulation execution unit 142. Further, the nonvolatile memory may be configured to store defaults of various data regarding cell proliferation. At least a part of the second storage unit 138 may be provided in the processor, the integrated circuit, and the like described above.

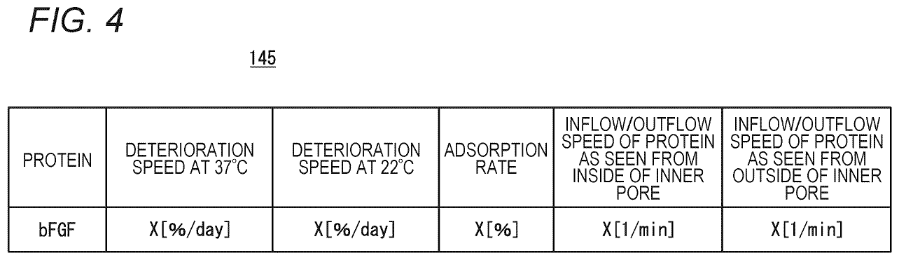

FIG. 4 is a table illustrating parameter information 145. The second storage unit 138 may be configured to store the parameter information 145. The parameter information 145 may include a data set of parameters used to perform an arithmetic operation regarding a protein in culture simulation. The parameter information 145 may include various types of information (“protein deterioration speed at 37° C.” (also referred to as deterioration rate, decomposition rate and the like), “protein deterioration speed at 22° C.” (also referred to as deterioration rate, decomposition rate and the like), “protein adsorption rate”, “inflow/outflow speed of protein as seen from inside of inner pore”, “inflow/outflow speed of protein as seen from outside of inner pore”, the like, or any combination thereof) corresponding to a protein type. In FIG. 4, the parameter information 145 of bFGF, which is one example growth factors, is illustrated. The second storage unit 138 be configured, additionally or alternatively, to store the parameter information 145 corresponding to proteins other than bFGF.

The “protein deterioration speed at 37° C.” is a protein deterioration speed caused by temperature around the first circulation flow path 58. This is a parameter on the premise that the first circulation flow path 58 is placed in a temperature environment at 37° C. When the first circulation flow path 58 is placed in another temperature environment, a parameter corresponding to the temperature environment may be set.

Similarly, the “protein deterioration speed at 22° C.” is a protein deterioration speed caused by temperature around the first supply unit 22a. This is a parameter on the premise that the first supply unit 22a is placed in a temperature environment at 22° C. When the first supply unit 22a is placed in another temperature environment, a parameter corresponding to the temperature environment may be set.

The display unit 134 may include a human-machine interface, such as a display. The display unit 134 may include, for example, a human-machine interface integrated with the input unit 130 like a touch panel. The display unit 134 may be configured to display various screens as further described below.

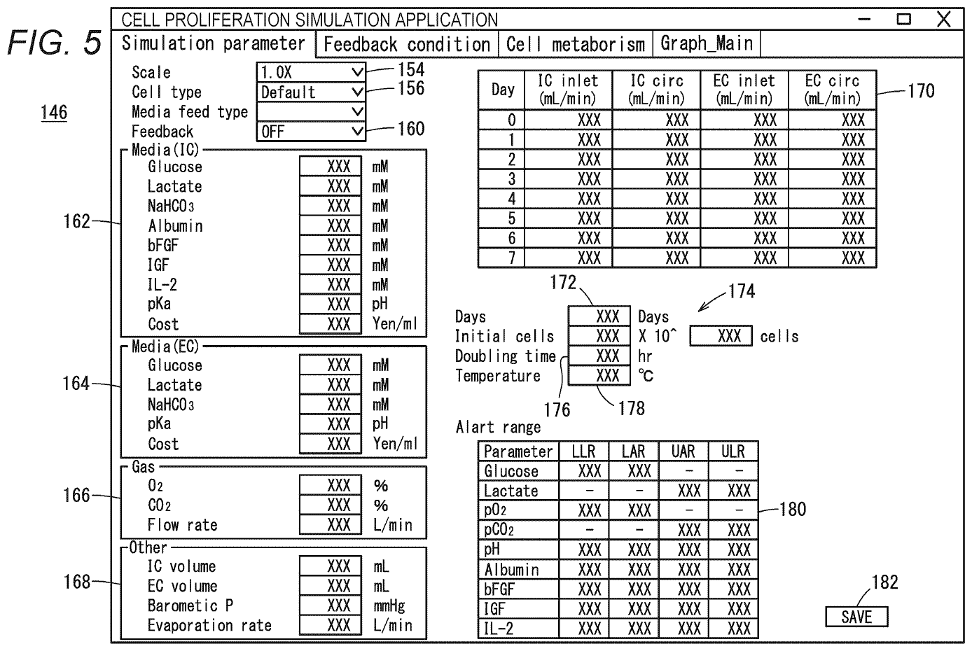

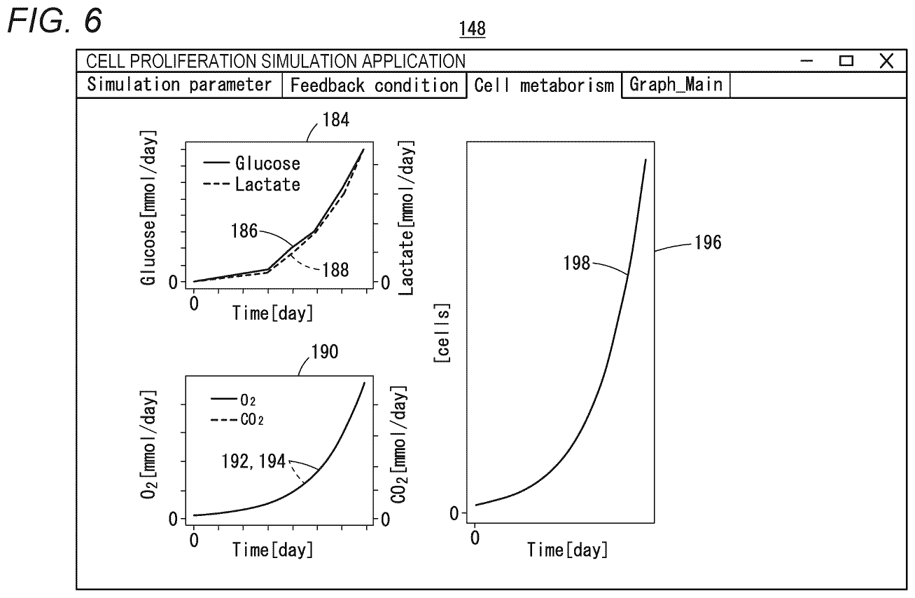

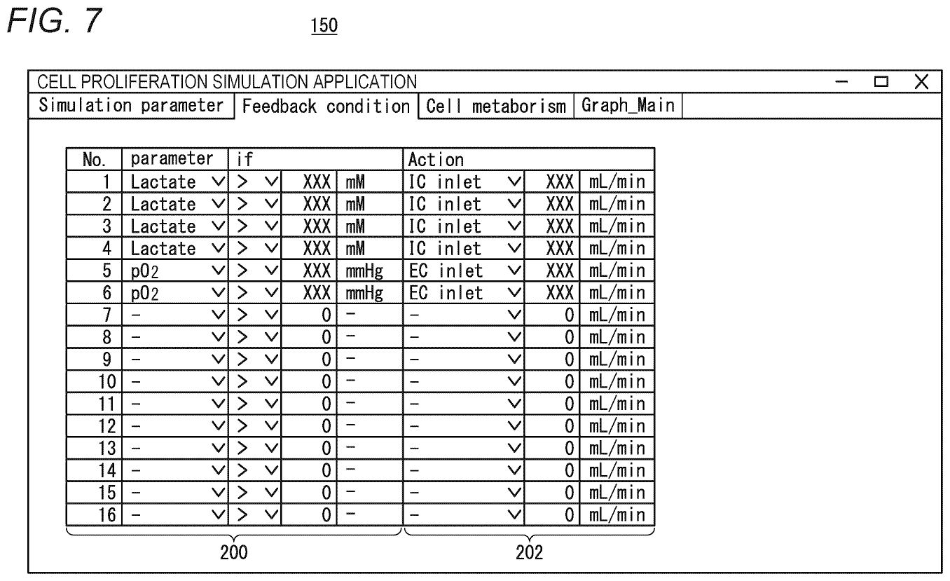

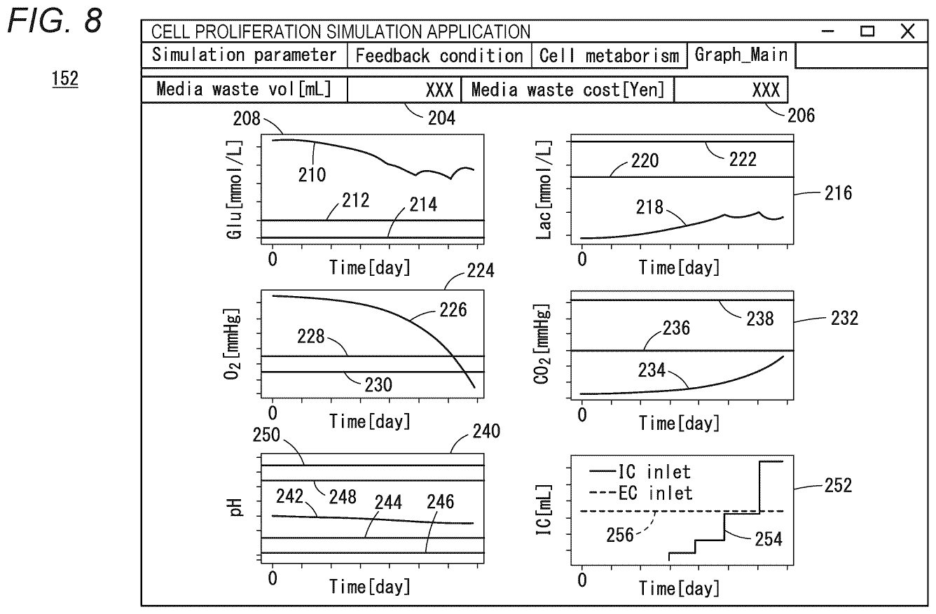

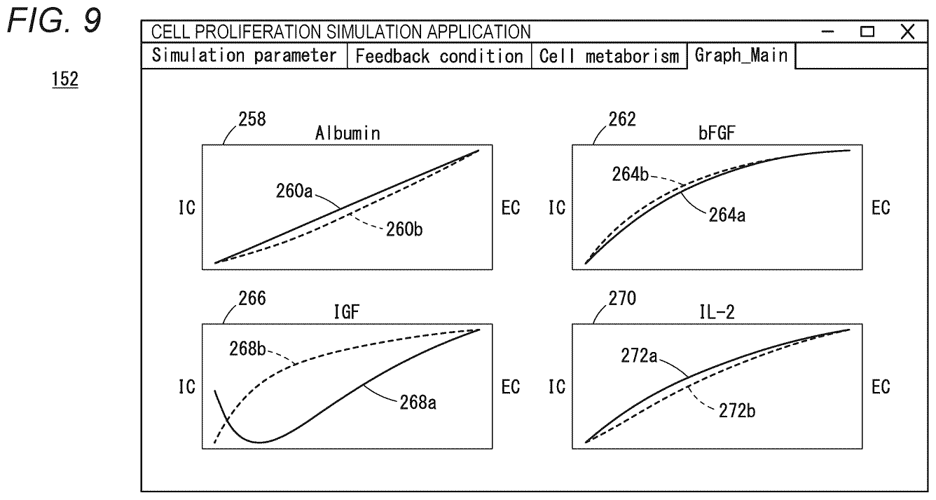

In at least one example embodiment, the display unit 134 may be configured display an input screen 146 (FIG. 5), a proliferation data screen 148 (FIG. 6), a feedback condition screen 150 (FIG. 7), a result screen 152 (FIGS. 8 and 9), the like, or any combination thereof.

FIG. 5 is a diagram illustrating the input screen 146 displayed on the display unit 134. The input screen 146 may be a screen for inputting various data used in cell culture simulation. When the user operates the input unit 130, the display unit 134 may display the input screen 146.

The input screen 146 may include a scale field 154. The scale field 154 may be an input field for designating a scale of cell culture in simulation. The user may select a scale from a drop-down list displayed in the scale field 154.

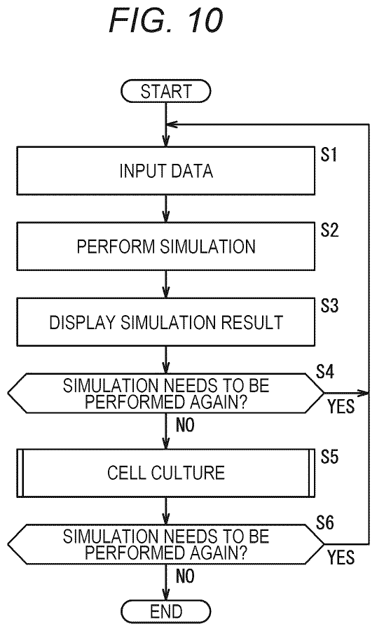

The input screen 146 may include a cell type field 156. The cell type field 156 may be an input field for designating proliferation data used in the simulation. The proliferation data may include data indicating a cell proliferation state under any culture condition. The proliferation data designated in the cell type field 156 may be a proliferation model of cells for simulation. The proliferation data may be created on the basis of data actually measured at a cell culture step performed in the past. The second storage unit 138 may be configured to store a default of the proliferation data. The second storage unit 138 may be configured to store data actually measured at the cell culture step illustrated at step S5 in FIG. 10 as the proliferation data. An example of the proliferation data is illustrated in FIG. 6. The user can select either the default or an actual measurement t result from a drop-down list displayed in the cell type field 156.

The input screen 146 may include a feedback field 160. The feedback field 160 may be an input field for designating whether to use a feedback condition in the simulation. A specific example of the feedback condition is illustrated in FIG. 7. The user can select either “ON” or “OFF” from a drop-down list displayed in the feedback field 160. When “ON” is selected, the feedback condition is used in the simulation. When “OFF” is selected, the feedback condition is not used in the simulation.

The input screen 146 may include a complete medium input field 162. The complete medium input field 162 may be an input field for designating data of the complete medium to be circulated in the first circulation flow path 58 in the simulation. Examples of the data of the complete medium may include the glucose concentration, the lactic acid concentration, the concentration of one or more proteins, the type of the complete medium, pka, a unit price of the complete medium, the like, or any combination thereof. The protein may be of one type or a plurality of types. The user can designate the protein to be added to the complete medium in the complete medium input field 162. The data of the complete medium may be condition data indicating a culture condition of the simulation.

The input screen 146 may include a basal medium input field 164. The basal medium input field 164 may be an input field f designating data of the basal medium to be circulated in the second circulation flow path 62 in the simulation. Examples of the data of the basal medium may include the glucose concentration, the lactic acid concentration, the type of the basal medium, pka, a unit price of the basal medium, the like, or any combination thereof. The data of the basal medium may be condition data indicating a culture condition of the simulation.

The input screen 146 may include a gas input field 166. The gas input field 166 may be an input field for designating data of the gas to be used in the gas exchange unit 34 in the simulation. Examples of the data of the gas may include a volume ratio of oxygen contained in the gas, a volume ratio of carbon dioxide contained in the gas, a flow rate of the gas, the like, or any combination thereof. The data of the gas may be condition data indicating a culture condition of the simulation.

The input screen 146 may include other input field 168. The other input field 168 may be an input field for designating other data regarding the culture medium. The other data may include a capacity of the first circulation flow path 58, a capacity of the second circulation flow path 62, an atmospheric pressure, a water vapor pressure, the like, or any combination thereof. The other data may be condition data indicating a culture condition of the simulation.

The input screen 146 may include a pump speed input field 170. The pump speed input field 170 may be an input field for designating a flow rate of each pump 98 in the simulation. The flow rate of each pump 98 may be set for each day of the culture period. The flow rate of each pump 98 may be condition data indicating a culture condition of the simulation.

The input screen 146 may include a day count input field 172. The day count input field 172 may be an input field for designating the number of culture days of the cells in the simulation. The number of culture days may be condition data indicating a culture condition of the simulation.

The input screen 146 may include a seeding times input field 174. The seeding times input field 174 may be an input field for designating the number of times of seeding in the simulation. The number of times of seeding may be condition data indicating a culture condition of the simulation.

The input screen 146 may include a doubling time input field 176. The doubling time input field 176 may be an input field for designating a time (doubling time) in which the cell doubles in the simulation. The doubling time may be condition data indicating a culture condition of the simulation.

The input screen 146 may include a temperature input field 178. The temperature input field 178 may be an input field for designating environmental temperature in the simulation. The environmental temperature may be condition data indicating a culture condition of the simulation.

The input screen 146 may include a threshold input field 180. The threshold input field 180 may be an input field for designating a threshold for each of the glucose concentration, the lactic acid concentration, a partial pressure of oxygen, a partial pressure of carbon dioxide, the pH, and protein. As the threshold, at least one of a lower limit value (LLR), a lower alert value (LAR), an upper limit value (ULR), and an upper alert value (UAR) is designated. For example, in at least one example embodiment, it is possible that only the lower limit value (LLR) and the lower alert value (LAR) are designated. For example, in at least one other example embodiment, it is possible that only the upper limit value (ULR) and the upper alert value (UAR) are designated. For example, in at least one other example embodiment, it is possible that the lower limit value (LLR), the lower alert value (LAR), the upper limit value (ULR), and the upper alert value (UAR) are designated. The user can optionally designate the threshold.

FIG. 6 is a diagram illustrating the proliferation data screen 148 displayed on the display unit 134. The proliferation data screen 148 may be a screen illustrating each piece of proliferation data. When the user operates the input unit 130, the display unit 134 may display the proliferation data screen 148. The second storage unit 138 may be configured to store each piece of proliferation data as a data set.

The proliferation data screen 148 may include a biodata graph 184. In the biodata graph 184, the horizontal axis represents time, and the vertical axis represents a metabolic speed of biodata. In the biodata graph 184, a metabolic speed line 186 and a metabolic speed line 188 are displayed. The metabolic speed line 186 indicates transition of the metabolic speed of glucose. The metabolic speed line 188 indicates transition of the metabolic speed of lactic acid. The metabolic speed of the biodata is the proliferation data indicating the cell proliferation state.

The proliferation data screen 148 may include a gas data graph 190. In the gas data graph 190, the horizontal axis represents time, and the vertical axis represents the metabolic speed of the biodata. In the gas data graph 190, a metabolic speed line 192 and a metabolic speed line 194 are displayed. The metabolic speed line 192 indicates transition of the metabolic speed of oxygen. The metabolic speed line 194 indicates transition of the metabolic speed of carbon dioxide. The metabolic speed of the gas data is the proliferation data indicating the cell proliferation state.

The proliferation data screen 148 may include a cell graph 196. In the cell graph 196, the horizontal axis represents time and the vertical axis represents the number of cells. In the cell graph 196, a cell count line 198 is displayed. The cell count line 198 indicates transition of the number of cells. The number of cells is the proliferation data indicating the cell proliferation state.

FIG. 7 is a diagram illustrating a feedback condition screen 150 displayed on the display unit 134. The feedback condition screen 150 may be a screen for inputting the feedback condition and change data. The feedback condition may be a condition for changing the condition data according to the situation of the simulation during the simulation. The change data may be a change value of the condition data. When the user operates the input unit 130, the display unit 134 may display the feedback condition screen 150.

The feedback condition screen 150 may include a condition field 200 and a data field 202. The condition field 200 may be an input field for designating the feedback condition. The data field 202 may be an input field for designating the change data. For example, the condition field 200 and the data field 202 indicated by No. 1 in FIG. 7 mean that “the flow rate of the first circulation pump 104 is set to XXX [mL/min] in a case where lactic acid is more than XXX [mM]”. The feedback condition and the change data may be the condition data indicating the culture condition of the simulation. Although not illustrated, it should be appreciated that, in various example embodiments, the feedback condition of glucose, carbon dioxide, pH, various proteins, the like, or any combination thereof can also be designated.

When a save button 182 on the input screen 146 illustrated in FIG. 5 is pressed, the second storage unit 138 may be configured to store data designated in each input field on the feedback condition screen 150.

FIGS. 8 and 9 are diagrams illustrating the result screen 152 displayed on the display unit 134. FIG. 8 illustrates an upper portion of the result screen 152, and FIG. 9 illustrates a lower portion of the result screen 152. When the user performs a downward scroll operation while the result screen 152 in FIG. 8 is displayed on the display unit 134, the result screen 152 in FIG. 9 is displayed on the display unit 134. The result screen 152 may be a screen illustrating a result of the simulation performed at step S2 in FIG. 10. After the simulation, when the user operates the input unit 130, the display control unit 144 may be configured to allow the display unit 134 to display the result screen 152.

The result screen 152 may include a discarded amount field 204 and a cost field 206. The total discarded amount of the culture medium in the simulated cell culture may be displayed in the discarded amount field 204. A cost in the simulated cell culture may be displayed in the cost field 206.

The result screen 152 may include a glucose graph 208 (FIG. 8). In the glucose graph 208, the horizontal axis represents time, and the vertical axis represents the glucose concentration. In the glucose graph 208, a concentration line 210, an alert line 212, and a lower limit line 214 may be displayed. The concentration line 210 may indicate transition of the glucose concentration during the culture period. The alert line 212 may indicate a boundary value between an OK range and an alert range. The lower limit line 214 may indicate a boundary value between the alert range and an NG range. The boundary value indicated by the alert line 212 may be a lower alert value of the glucose concentration input in the threshold input field 180 of the input screen 146. The boundary value indicated by the lower limit line 214 may be a lower limit value of the glucose concentration input in the threshold input field 180 of the input screen 146. A range above the alert line 212 may be the OK range. A range below the lower limit line 214 may be the NG range. A range between the alert line 212 and the lower limit line 214 may be the alert range. The concentration line 210 may be preferably in the OK range above the alert line 212. That is, the glucose concentration may be preferably within the OK range at all times during the culture period.

The result screen 152 may include a lactic acid graph 216 (FIG. 8). In the lactic acid graph 216, the horizontal axis represents time, and the vertical axis represents the lactic acid concentration. In the lactic acid graph 216, a concentration line 218, an alert line 220, and an upper limit line 222 may be displayed. The concentration line 218 may indicate transition of the lactic acid concentration during the culture period. The alert line 220 may indicate a boundary value between an OK range and an alert range. The upper limit line 222 may indicate a boundary value between the alert range and an NG range. The boundary value indicated by the alert line 220 may be an upper alert value of the lactic acid concentration input in the threshold input field 180 of the input screen 146. The boundary value indicated by the upper limit line 222 may be an upper limit value of the lactic acid concentration input in the threshold input field 180 of the input screen 146. A range below the alert line 220 may be an OK range. A range above the upper limit line 222 may be an NG range. A range between the alert line 220 and the upper limit line 222 may be an alert range. The concentration line 218 may be preferably in the OK range below the alert line 220. That is, the lactic acid concentration may be preferably within the OK range at all times during the culture period.

The result screen 152 may include an O₂ graph 224 (FIG. 8). In the O₂ graph 224, the horizontal axis represents time, and the vertical axis represents a partial pressure of oxygen. In the O₂ graph 224, a partial pressure line 226, an alert line 228, and a lower limit line 230 may be displayed. The partial pressure line 226 may indicate transition of the partial pressure of oxygen during the culture period. The alert line 228 may indicate a boundary value between an OK range and an alert range. The lower limit line 230 may indicate a boundary value between the alert range and an NG range. The boundary value indicated by the alert line 228 may be a lower alert value of the partial pressure of oxygen input in the threshold input field 180 of the input screen 146. The boundary value indicated by the lower limit line 230 may be a lower limit value of the partial pressure of oxygen input in the threshold input field 180 of the input screen 146. A range above the alert line 228 may be an OK range. A range below the lower limit line 230 may be an NG range. A range between the alert line 228 and the lower limit line 230 may be an alert range. The partial pressure line 226 may be preferably in the OK range above the alert line 228. That is, the partial pressure of oxygen may be preferably within the OK range at all times during the culture period.

The result screen 152 may include a CO₂ graph 232 (FIG. 8). In the CO₂ graph 232, the horizontal axis represents time, and the vertical axis represents the partial pressure of carbon dioxide. In the CO₂ graph 232, a partial pressure line 234, an alert line 236, and an upper limit line 238 may be displayed. The partial pressure line 234 may indicate transition of the partial pressure of carbon dioxide during the culture period. The alert line 236 may indicate a boundary value between an OK range and an alert range. The upper limit line 238 may indicate a boundary value between the alert range and an NG range. The boundary value indicated by the alert line 236 may be an upper alert value of partial pressure of carbon dioxide input in the threshold input field 180 of the input screen 146. The boundary value indicated by the upper limit line 238 may be an upper limit value of the partial pressure of carbon dioxide input in the threshold input field 180 of the input screen 146. A range below the alert line 236 may be an OK range. A range above the upper limit line 238 may be an NG range. A range between the alert line 236 and the upper limit line 238 may be an alert range. The partial pressure line 234 may be preferably in the OK range below the alert line 236. That is, the partial pressure of carbon dioxide may be preferably within the OK range at all times during the culture period.

The result screen 152 may include a pH graph 240 (FIG. 8). In the pH graph 240, the horizontal axis represents time, and the vertical axis represents the pH of the culture medium. In the pH graph 240, a pH line 242, a lower alert line 244, a lower limit line 246, an upper alert line 248, and an upper limit line 250 may be displayed. The pH line 242 may indicate transition of the pH during the culture period. The lower alert line 244 may indicate a boundary value between an OK range and a lower alert range. The lower limit line 246 may indicate a boundary value between the lower alert range and a first NG range. The upper alert line 248 may indicate a boundary value between the OK range and an upper alert range. The upper limit line 250 may indicate a boundary value between the upper alert range and a second NG range. The boundary value indicated by the lower alert line 244 may be a lower alert value of the pH input in the threshold input field 180 of the input screen 146. The boundary value indicated by the lower limit line 246 may be a lower limit value of the pH input in the threshold input field 180 of the input screen 146. The boundary value indicated by the upper alert line 248 may be an upper alert value of the pH input in the threshold input field 180 of the input screen 146. The boundary value indicated by the upper limit line 250 may be an upper limit value of the pH input in the threshold input field 180 of the input screen 146. A range between the lower alert line 244 and the upper alert line 248 may be an OK range. A range below the lower limit line 246 may be the first NG range. A range between the lower alert line 244 and the lower limit line 246 may be a lower alert range. The range above the upper limit line 250 may be the second NG range. A range between the upper alert line 248 and the upper limit line 250 may be the upper alert range. The pH line 242 may be preferably in the OK range between the lower alert line 244 and the upper alert line 248. That is, the pH of the culture medium may be preferably within the OK range at all times during the culture period.

The result screen 152 may include a flow rate graph 252 (FIG. 8). In the flow rate graph 252, the horizontal axis represents time, and the vertical axis represents the flow rate of the first circulation pump 104 and the flow rate of the second circulation pump 108. In the flow rate graph 252, a first flow rate line 254 and a second flow rate line 256 may be displayed. The first flow rate line 254 may indicate transition of the flow rate of the first circulation pump 104 during the culture period. The second flow rate line 256 may indicate transition of the flow rate of the second circulation pump 108 during the culture period.

The result screen 152 may include an albumin graph 258 (FIG. 9). In the albumin graph 258, the horizontal axis represents time and the vertical axis represents concentration. The left vertical axis (IC) represents the concentration in the first circulation flow path 58, and the right vertical axis (EC) represents an albumin concentration in the second circulation flow path 62. Concentration lines 260a and 260b are displayed in the albumin graph 258. The concentration line 260a may indicate transition of the albumin concentration in the first circulation flow path 58 during the culture period. The concentration line 260b may indicate transition of the albumin concentration in the second circulation flow path 62 during the culture period. Although not illustrated, it should be appreciated that, in various other example embodiments, a lower alert line, a lower limit line, an upper alert line, an upper limit line, the like, or any combination thereof may also be illustrated in the albumin graph 258 similarly to each graph in FIG. 8.

The result screen 152 may include a bFGF graph 262 (FIG. 9). In the bFGF graph 262, concentration lines 264a and 264b are displayed. The concentration line 264a may indicate transition of a bFGF concentration in the first circulation flow path 58 during the culture period. The concentration line 264b may indicate transition of the bFGF concentration in the second circulation flow path 62 during the culture period.

The result screen 152 may include an IGF graph 266 (FIG. 9). In the IGF graph 266, concentration lines 268a and 268b are displayed. The concentration line 268a may indicate transition of an IGF concentration in the first circulation flow path 58 during the culture period. The concentration line 268b may indicate transition of the IGF concentration in the second circulation flow path 62 during the culture period.

The result screen 152 may include an IL-2 graph 270 (FIG. 9). In the IL-2 graph 270, concentration lines 272a and 272b are displayed. The concentration line 272a may indicate transition of an IL-2 concentration in the first circulation flow path 58 during the culture period. The concentration line 272b may indicate transition of the IL-2 concentration in the second circulation flow path 62 during the culture period.

The result screen 152 may illustrate transition of concentrations of other proteins. The result screen 152 may illustrate transition of the concentration of the protein designated in the complete medium input field 162 of the input screen 146.

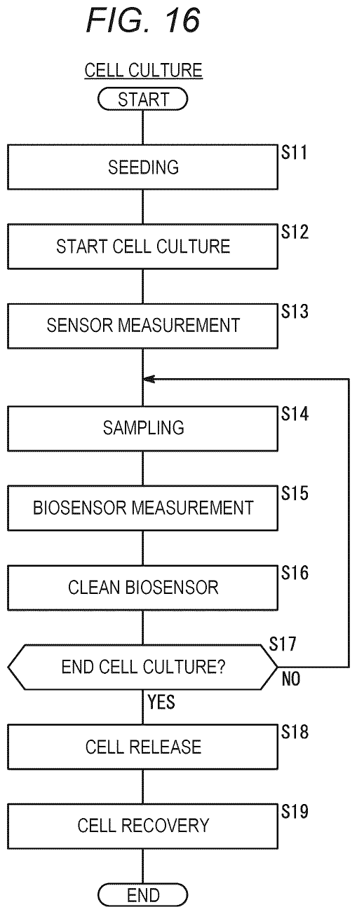

FIG. 10 is a flowchart illustrating a flow of a cell culture method performed using the cell culture system 10. Steps S1 to S3 in FIG. 10 may be performed by the simulation apparatus 14. Step S5 in FIG. 10 may be performed by the cell culture apparatus 12. Step S5 is illustrated in detail in FIG. 16. Steps S4 and S6 are determined by the user.

Before step S1, the user may interact with the input unit 130 to start the simulation program. The second arithmetic unit 136 may be configured to execute the simulation program stored in the second storage unit 138 in response to the operation of the user. The display control unit 144 may be configured to allow the display unit 134 to display the input screen 146 illustrated in FIG. 5.

At step S1, the user may interact with the input unit 130 to input data in each input field of the input screen 146. For example, in initial simulation, the user may designate default in the cell type field 156. The user may press the save button 182 after designating each piece of data. The input unit 130 may be configured to input data in each input field to the simulation unit 132. The second storage unit 138 may be configured to store each piece of data. When step S1 ends, the processing proceeds to step S2.

At step S2, the user may interact with the input unit 130 to start cell culture simulation. In response to the instruction from the input unit 130, the simulation execution unit 142 may be configured to start the cell culture simulation using each piece of data (e.g., proliferation data, condition data, various parameters of protein, or any combination thereof) stored in the second storage unit 138. The simulation execution unit 142 may be configured to simulate the cell culture during a designated culture period using the proliferation data, the condition data, and various parameters of protein. The simulation execution unit 142 may be configured to calculate the amount of each component in the culture medium at each time during the culture period. For example, the simulation execution unit 142 may be configured to calculate the glucose concentration at each time during the culture period. The simulation execution unit 142 may be configured to calculate the lactic acid concentration at each time during the culture period. The simulation execution unit 142 may be configured to calculate the partial pressure of oxygen at each time during the culture period. The simulation execution unit 142 may be configured to calculate the partial pressure of carbon dioxide at each time during the culture period. The simulation execution unit 142 may be configured to calculate the pH of the culture medium at each time during the culture period. The simulation execution unit 142 may be configured to calculate the amount of each component by a known arithmetic method. The arithmetic method is detailed, for example, in the literature “Journal of Chemical Technology and Metallurgy, 48, 4, 2013, 351-356 EXPERIMENTAL DETERMINATION OF THE VOLUMETRIC MASS TRANSFER COEFFICIENT”, the entire contents of which are hereby incorporated by reference. The simulation execution unit 142 may be configured to calculate the concentration of protein at each time during the culture period. The second storage unit 138 may be configured to store an arithmetic result of the simulation execution unit 142.

The simulation execution unit 142 may be configured to determine whether the feedback condition is satisfied on the basis of each arithmetic value at each time. In a case where the feedback condition is satisfied, the simulation execution unit 142 may be configured to change a part of the culture condition according to setting of the feedback condition. For example, the simulation execution unit 142 may be configured to change the data of the flow rate of any one of the pumps 98. The simulation execution unit 142 may be configured to continue the simulation using the changed data. The second storage unit 138 may be configured to store the changed condition data.

When the simulation of the cell culture ends, the simulation execution unit 142 may be configured to calculate the total consumption amount and the total discarded amount of the culture medium in the simulation. The simulation execution unit 142 may be configured to calculate a cost by using the total consumption amount of the culture medium and the unit price of the culture medium. The second storage unit 138 may be configured to store an arithmetic result of the simulation execution unit 142. When step S2 ends, the processing proceeds to step S3.

At step S3, the user may interact with the input unit 130 to display the result of the simulation. In response to the instruction from the input unit 130, the display control unit 144 may be configured to allow the display unit 134 to display the result of the simulation. The display unit 134 may be configured to display the result screen 152 illustrated in FIGS. 8 and 9. When step S3 ends, the processing proceeds to step S4.

At step S4, the user may determine whether the simulation needs to be performed again. In any graph of the result screen 152, when there is a part deviating from the OK range in the transition of the arithmetic value, the user may change the condition data and again execute the simulation. When the simulation needs to be performed again (step S4: YES), the processing may return to step S1. In contrast, when the simulation does not need to be performed again (step S4: NO), the processing may proceed to step S5.

At step S5, the user may perform the cell culture using the cell culture apparatus 12. The user may interact with an input device (not illustrated) of the cell culture apparatus 12 to set the culture condition designated at step S1 in FIG. 10. For example, when the control unit 20 of the cell culture apparatus 12 and the simulation unit 132 of the simulation apparatus 14 are connected by a signal line, the control unit 20 may be configured to acquire the condition data of the culture condition from the second storage unit 138 of the simulation unit 132. After the processing of the cell culture ends, the simulation unit 132 may be configured to acquire the condition data of the culture condition and the proliferation data of a new cell from the control unit 20. The second storage unit 138 may be configured to store each piece of data acquired from the control unit 20. When step S5 ends, the processing proceeds to step S6.

At step S6, the user may determine whether the simulation needs to be performed again. The cell culture may be performed a plurality of times. The user may increase the scale of the cell culture stepwise as the number of times of cell culture increases. The user may perform the simulation each time the scale of the cell culture is increased. When the simulation needs to be performed again (step S6: YES), the processing may return to step S1. In contrast, in a case where the simulation does not need to be performed again (step S6: NO), the cell culture may end.

At step S6, the user may compare the result of the simulation performed at step S3 with the measurement result in the cell culture performed at step S5. For example, the display control unit 144 may allow the display unit 134 to display a predicted value of the protein and the like obtained by the simulation and an actually measured value of the protein and the like obtained by actual cell culture. Further, the display control unit 144 may allow the display unit 134 to display a range of ±5%×day with respect to the actually measured value as an allowable range of the protein and the like. For example, the display control unit 144 may be configured to calculate a value of −58×day with respect to the actually measured value and to set the same as a lower limit value of the allowable range. The display control unit 144 may be configured to calculate a value of +5%×day with respect to the actually measured value and to set the same as an upper limit value of the allowable range.

The simulation execution unit 142 may be configured to calculate the transition of the concentration of protein during the designated culture period. The simulation execution unit 142 may be configured to calculate the concentration every predetermined time as the transition of the concentration of protein. For example, the simulation execution unit 142 may be configured to calculate the concentration at one-minute intervals. An interval at which the concentration of protein is calculated is not limited thereto. For example, the interval at which the concentration of protein is calculated may be a one-second interval or a one-day interval.

The simulation execution unit 142 may be configured to calculate the transition of the concentration of protein by the calculation method according to the culture form in which the simulation is executed. For example, the simulation execution unit 142 may configured to execute simulations in eight culture forms (first to eighth culture forms) as follows: the first culture form, the third culture form, the fifth culture form, and the seventh culture form may be executed in a culture process of floating cells; And the second culture form, the fourth culture form, the sixth culture form, and the eighth culture form may be executed in a culture process of adherent cells. In each culture process of the floating cells and the adherent cells, one culture form among the plurality of culture forms may be executed or a plurality of culture forms may be combined.

The first culture form and the second culture form may be mainly executed from an initial stage to a middle stage of the cell culture process. The third culture form and the fourth culture form may be mainly executed from the middle stage to a later stage of the cell culture process. The fifth culture form may be appropriately executed during the culture period in order to collect cells. Each culture form may be different in a supplied culture medium, a method of supplying the culture medium, a method of discarding the culture medium, the like, or any combination thereof. The simulation execution unit 142 may be configured to execute the simulation obtained by combining a plurality of culture forms in a case of executing the simulation of culture in any period.

The simulation execution unit 142 may be configured to use each of a calculation expression of the protein concentration in the first circulation flow path 58 and a calculation expression of the protein concentration in the second circulation flow path 62 determined in each culture form and connect the data obtained by calculating the protein concentration in each culture period by each culture form to obtain the simulation result of the entire culture period. For example, the simulation execution unit 142 may be configured to calculate data indicating the concentration for each culture period by using the calculation expression of the protein concentration of the first culture form, the third culture form, and the fifth culture form performed in the culture of the floating cells and connect the calculated data to thereby obtain the simulation result of the entire culture period. The simulation execution unit 142 may be configured to calculate data indicating the concentration for each culture period by using the calculation expression of the protein concentration of the second culture form and the fourth culture form performed in the culture of the adherent cells and connect the calculated data to thereby obtain the simulation result of the entire culture period.

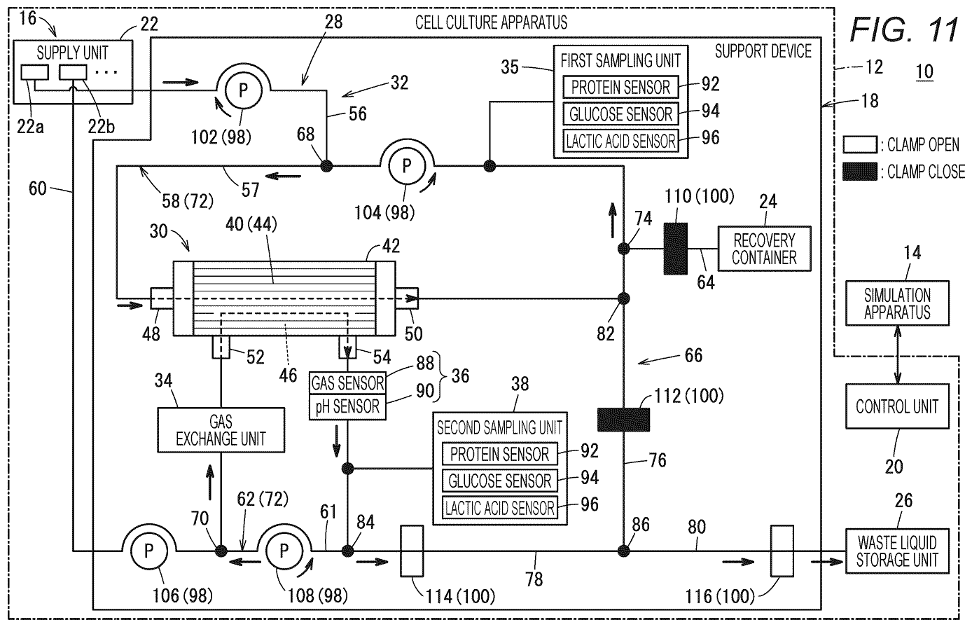

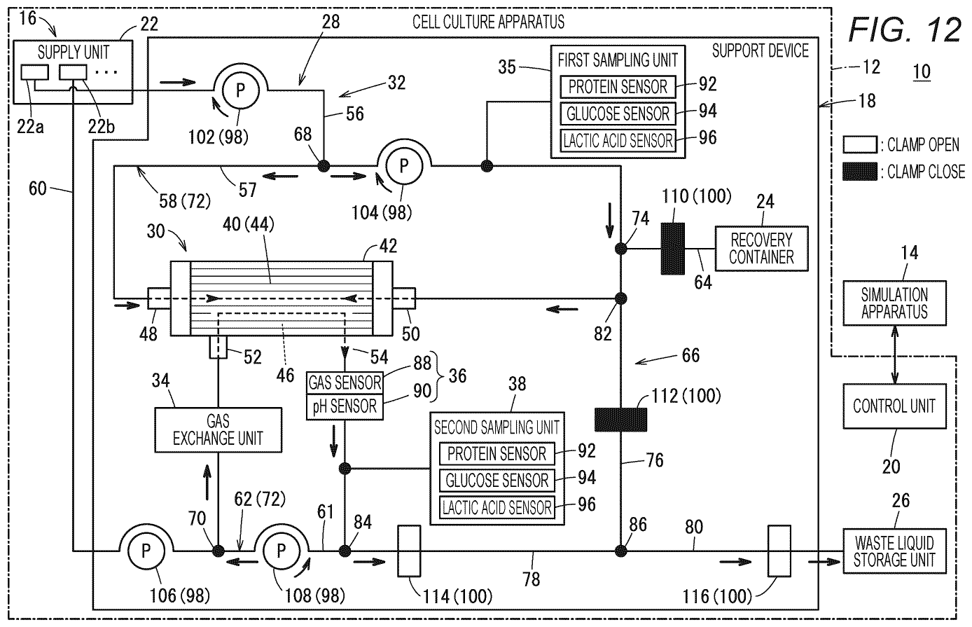

FIG. 11 is a diagram illustrating an operation of the cell culture apparatus 12 at the time of cell culture in the first culture form. FIG. 12 is a diagram illustrating an operation of the cell culture apparatus 12 at the time of cell collection in the first culture form. In the first culture form, the control unit 20 may be configured to control the first supply unit 22a, the second supply unit 22b, each pump 98, and each clamp 100 to adjust the flow path through which the culture medium flows and the flow rate of the culture medium. Similarly for the second to eighth culture forms, the control unit 20 may be configured to adjust the flow path through which the culture medium flows and the flow rate of the culture medium. The first culture form may be as follows: (a) the complete medium is supplied from the first supply unit 22a to the first circulation flow path 58; (b) the basal medium is not supplied from the second supply unit 22b to the second circulation flow path 62; and (c) a part of the culture medium is discarded from the second circulation flow path 62 to the waste liquid storage unit 26 via the second waste liquid flow path 78.

The simulation execution unit 142 may be configured to calculate the concentration of protein (Pro_IC [n+1]) in the first circulation flow path 58 using formulas (1) to (5). The simulation execution unit 142 may be configured to calculate the concentration of protein (Pro_EC [n+1]) in the second circulation flow path 62 using formulas (6) to (9).

$$\frac{Pro\_IC [ n + 1 ] - Pro\_IC [ n ]}{t [ n + 1 ] - t [ n ]} = F\_i \_b + L\_b \_c + E\_t \_m1 + L\_i \_b1 \qquad Formula ( 1 )$$

$$F\_i \_b = \frac{C m \times ( 1 - A\_r ) \times ( 1 - Deg\_ 22 \times t\_ [ n ] ) \times IC\_ir}{V\_IC} \qquad Formula ( 2 )$$

$$L\_b \_c = \frac{Mx\_pro [ n ]}{V\_IC} \qquad Formula ( 3 )$$

$$E\_t \_m1 = k\_Etol \times ( Pro\_EC [ n ] - Pro\_IC [ n ] ) \qquad Formula ( 4 )$$

$$L\_i \_b1 = Pro\_IC [ n ] \times ( - Deg\_ 37 ) \qquad Formula ( 5 )$$

$$\frac{Pro\_EC [ n + 1 ] - Pro\_EC [ n ]}{t [ n + 1 ] - t [ n ]} = L\_t \_w2 + E\_t \_m2 + L\_i \_b2 \qquad Formula ( 6 )$$

$$L\_t \_w2 = - \frac{Pro\_EC [ n ] \times IC\_ir}{V\_EC} \qquad Formula ( 7 )$$

$$E\_t \_m2 = k\_ItoE \times ( Pro\_IC [ n ] - Pro\_EC [ n ] ) \qquad Formula ( 8 )$$

$$L\_i \_b2 = Pro\_EC [ n ] \times ( - Deg\_ 37 ) \qquad Formula ( 9 )$$

The variables included in expressions (1) to (9) above are defined as follows:
[n] is a variable obtained in an n-th calculation among calculations performed at one-minute intervals.
n is a numerical value from zero to (length of culture period/interval of calculation time). Pro_IC: concentration of protein in first circulation flow path 58 [unit: ng/mL]. Pro_EC: concentration of protein in second circulation flow path 62 [unit: ng/mL]. t: elapsed time in simulation [unit: min]. Cm: concentration of protein in complete medium supplied from first supply unit 22a to first circulation flow path 58 [unit: ng/mL]. A_r: rate at which protein disappears from culture medium of first supply flow path 56 and first circulation flow path 58 due to protein adsorption, lamination and the like on inner surface of hollow fiber membrane 40 [unit: %]. Deg_22: protein deterioration rate (disappearance rate) caused by ambient temperature (22° C.) in first supply unit 22a [%/day]. Deg_37: protein deterioration rate (disappearance rate) caused by ambient temperature (37° C.) in first circulation flow path 58 [%/day]. IC_ir: flow rate of first supply pump 102 [unit: mL/min]. V_IC: volume of first circulation flow path 58 [unit: mL]. V_EC: volume of second circulation flow path 62 [unit: mL]. Mx_pro: consumption speed per unit time of protein consumed by cells [unit: mg/min]. k_EtoI: speed (coefficient) at which protein in culture medium flows in and out between inside and outside of inner pore via hollow fiber membrane 40 as seen from inside of inner pore [unit: 1/min]. k_ItoE: speed (coefficient) at which protein in culture medium flows in and out between inside and outside of inner pore via hollow fiber membrane 40 as seen from outside of inner pore [unit: 1/min].

A_r is also referred to as an adsorption rate or a correction rate. In other words, the adsorption rate (correction rate) is a rate of protein that cannot be consumed by cells because protein is adsorbed, aggregated, and deposited on at least one of the inside of the first circulation flow path (inner circulation path) 58 and the inside of the second circulation flow path (outer circulation path) 62.

Each item included in formula (1) is defined as follows:
Pro_IC[n+1]−Pro_IC[n]/t[n+1]−t[n]: change speed of concentration of protein in first circulation flow path 58 between timing of n-th calculation and timing of (n+1)-th calculation. F_i_b: speed (supply speed) at which concentration of protein changes in first circulation flow path 58 due to supply of culture medium to first circulation flow path 58. L_b_c: speed (consumption speed) at which concentration of protein changes in first circulation flow path 58 due to consumption of protein by cells. E_t_m1: speed (exchange speed) at which concentration of protein changes in first circulation flow path 58 due to inflow and outflow of culture medium between inside and outside of inner pore via hollow fiber membrane 40. For this concentration, the volume of the first circulation flow path 58 is used as a denominator. L_i_b1: speed (deterioration speed) at which concentration of protein changes in first circulation flow path 58 due to protein deterioration in first circulation flow path 58.

Each item included in formula (6) is defined as follows:
Pro_EC[n+1]−Pro_EC[n]/t[n+1]−t[n]: change speed of concentration of protein in second circulation flow path 62 between timing of n-th calculation and timing of (n+1)-th calculation. L_t_w2: speed (discard speed) at which the concentration of protein changes in second circulation flow path 62 by discarding a part of culture medium via second circulation flow path 62. E_t_m2: speed (exchange speed) at which concentration of protein changes in second circulation flow path 62 due to inflow and outflow of culture medium between inside and outside of inner pore via hollow fiber membrane 40. For this concentration, the volume of the second circulation flow path 62 is used as a denominator. L_i_b2: speed (deterioration speed) at which concentration of protein changes in second circulation flow path 62 due to protein deterioration in second circulation flow path 62.

The acquisition unit 140 may be configured to acquire Cm of expression (2) above, IC_ir of expressions (2) and (7) above, V_IC of expressions (2) and (3) above, and V_EC of formula (7) from the information input to the input screen 146. The acquisition unit 140 may be configured to acquire A_r of formula (2), Deg_22 of formula (2), k_EtoI of formula (4), Deg 37 of formulas (5) and (9), and k_ItoE of formula (8) from the parameter information 145 stored in the second storage unit 138. The acquisition unit 140 may be configured to acquire Pro_IC[n] and Pro_EC[n] from the previous calculation result. Pro_IC[0] and Pro_EC[0] are initial values of concentration of protein. The calculation result of each time may be stored in the second storage unit 138. The acquisition unit 140 may be configured to acquire Mx_pro[n] from the second storage unit 138. In at least one example embodiment, Mx_pro[n] may be zero.

The simulation execution unit 142 may be configured to calculate Pro_IC[n+1] and Pro_EC[n+1] as a result of the (n+1)-th calculation by substituting each parameter acquired by the acquisition unit 140 into formulas (1) to (9).

The first culture form may be primarily executed in the cell culture process but may also be executed in a cell collection process. When the first culture form is executed in the cell collection process, for example, as illustrated in FIG. 12, the basal medium may be supplied in both directions of the first inlet port 48 and the first outlet port 50 starting from the first junction 68. As a result, the cells present in the first circulation flow path 58 may be collected in the hollow fiber membrane 40. The calculation expression of the concentration of protein in the first culture form may be the same in the cell culture process and the cell collection process.

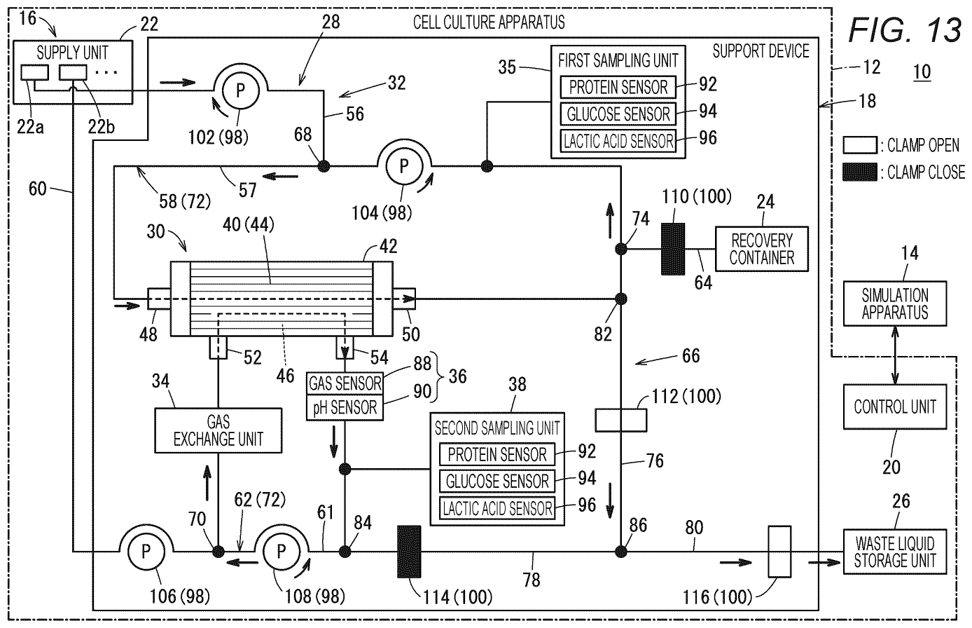

FIG. 13 is a diagram illustrating an operation of the cell culture apparatus 12 at the time of cell culture in the second culture form. The second culture form may be as follows: (a) the complete medium is supplied from the first supply unit 22a to the first circulation flow path 58; (b) the basal medium is not supplied from the second supply unit 22b to the second circulation flow path 62; and (c) a part of the culture medium is discarded from the first circulation flow path 58 to the waste liquid storage unit 26 via the first waste liquid flow path 76.

In the second culture form, formulas (2) to (5), formula (8), and formula (9) may be used. In the second culture form, the following formulas (10) and (11) may be used instead of formula (1). In the second culture form, the following formula (12) may be used instead of expression (6) above.

$$\frac{Pro\_IC [ n + 1 ] - Pro\_IC [ n ]}{t [ n + 1 ] - t [ n ]} = L\_t \_w1 + F\_i \_b + L\_b \_c + E\_t \_m1 + L\_i \_b1 \qquad Formula ( 10 )$$

$$L\_t \_w1 = - \frac{Pro\_IC [ n ] \times IC\_ir}{V\_IC} \qquad Formula ( 11 )$$

$$\frac{Pro\_EC [ n + 1 ] - Pro\_EC [ n ]}{t [ n + 1 ] - t [ n ]} = E\_t \_m2 + L\_i \_b2 \qquad Formula ( 12 )$$

An item of formula (11) is defined as follows:
L_t_w1: speed (discard speed) at which concentration of protein changes in first circulation flow path 58 by discarding a part of culture medium via first circulation flow path 58.

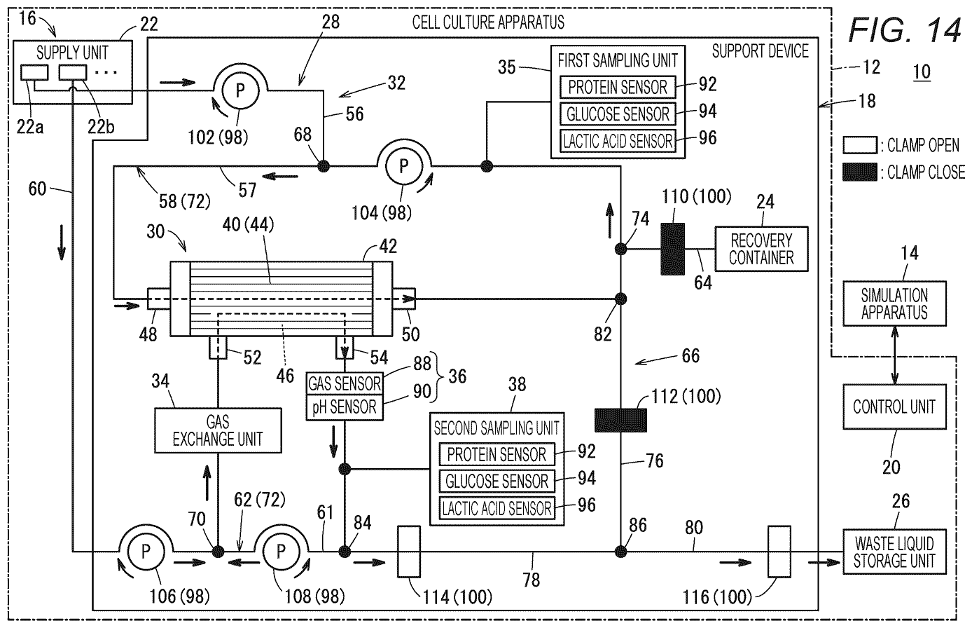

FIG. 14 is a diagram illustrating an operation of the cell culture apparatus 12 at the time of cell culture in the third culture form. The third culture form may be as follows: (a) the complete medium is supplied from the first supply unit 22a to the first circulation flow path 58; (b) the basal medium is supplied from the second supply unit 22b to the second circulation flow path 62; and (c) a part of the culture medium is discarded from the second circulation flow path 62 to the waste liquid storage unit 26 via the second waste liquid flow path 78.

In the third culture form, formulas (1) to (6), formula (8) above, and formula (9) may be used. In the third culture form, the following formula (13) may be used instead of formula (7).

$$L\_t \_w2 = - \frac{Pro\_EC [ n ] \times ( IC\_ir + EC\_ir )}{V\_EC} \qquad Formula ( 13 )$$

An item of formula (13) is defined as follows:
EC_ir: flow rate of second supply pump 106 [unit: mL/min].

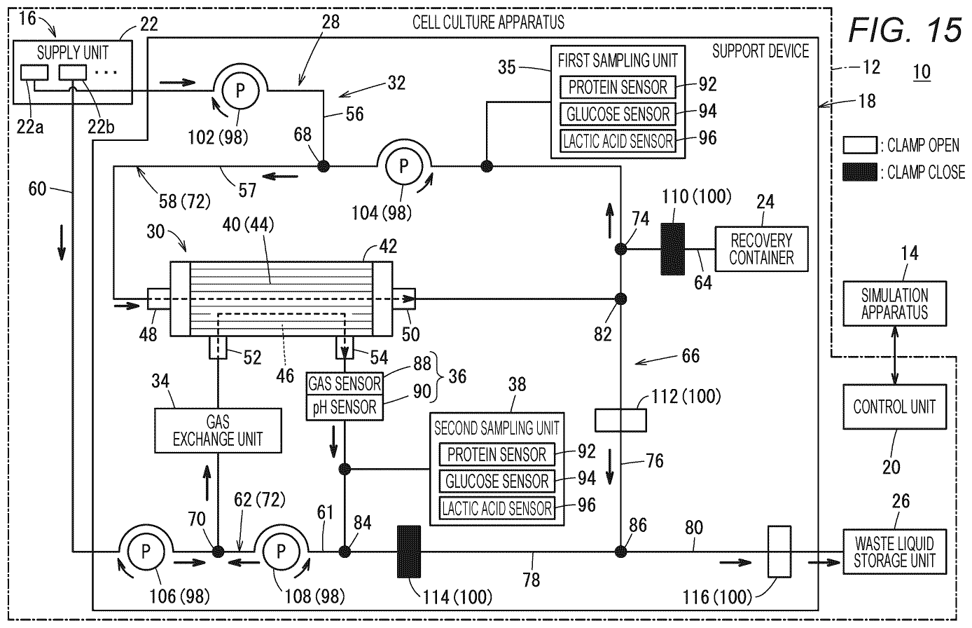

FIG. 15 is a diagram illustrating an operation of the cell culture apparatus 12 at the time of cell culture in the fourth culture form. The fourth culture form may be as follows: (a) the complete medium is supplied from the first supply unit 22a to the first circulation flow path 58; (b) the basal medium is supplied from the second supply unit 22b to the second circulation flow path 62; and (c) a part of the culture medium is discarded from the first circulation flow path 58 to the waste liquid storage unit 26 via the first waste liquid flow path 76.

In the fourth culture form, formulas (2) to (5), formula (8) above, and formula (9) may be used. In the fourth culture form, formula (10) and the following formulas (14) may be used instead of formula (1) above. In the fourth culture form, expression (12) may be used instead of expression (6).

$$L\_t \_w1 = - \frac{Pro\_IC [ n ] \times ( IC\_ir + EC\_ir )}{V\_IC} \qquad Formula ( 14 )$$

The fifth culture form may be as follows: (a) the basal medium is supplied from the first supply unit 22a to the first circulation flow path 58; (b) the basal medium is not supplied from the second supply unit 22b to the second circulation flow path 62; and (c) a part of the culture medium is discarded from the second circulation flow path 62 to the waste liquid storage unit 26 via the second waste liquid flow path 78. That is, the operation of the cell culture apparatus 12 at the time of cell culture in the fifth culture form may be the same as the operation of the cell culture apparatus 12 at the time of cell culture in the first culture form (FIGS. 11 and 12) except for (a).

In the fifth culture form, as in the first culture form, formulas (1) to (9) may be used. However, in the fifth culture form, since the basal medium is used instead of the complete medium, protein is not supplied from the first supply unit 22a to the first circulation flow path 58. Therefore, Cm in formula (2) is zero. That is, formula (2) above becomes the following formula (2)′.

$$F\_i \_b = 0 \qquad Formula {( 2 )}^{′}$$

A reason for supplying the basal medium to the first circulation flow path 58 may be to remove waste products such as lactic acid. As cells proliferate, the waste products (such as lactic acid) accumulate in the first region 44 that is the inner pore of the hollow fiber membrane 40. For example, as in the second culture form, it may be possible to sweep away the waste products from the inside to the outside of the first region 44 by supplying the complete medium to the first circulation flow path 58. However, when the complete medium is used for removing the waste products, protein may be discarded together with the waste products. Therefore, the discarded protein is wasted. By using the basal medium instead of the complete medium, the waste products may be removed from the first region 44 while preventing the protein from being used more than necessary.

In the fifth culture form, the waste products accumulated in the first region 44 may be pushed out to the second region 46 through the pore of the hollow fiber membrane 40. The waste products in the second region 46 may be discarded together with the culture medium into the waste liquid storage unit 26 via the second circulation flow path 62. According to the fifth culture form, the waste products accumulated in the first region 44 may be discarded into the waste liquid storage unit 26 without significantly changing the concentration of protein in the first circulation flow path 58.

In the fifth culture form, similarly to the first culture form, the basal medium may be supplied in both directions of the first inlet port 48 and the first outlet port 50 starting from the first junction 68. As a result, the cells present in the first circulation flow path 58 may be collected in the hollow fiber membrane 40. A step in the fifth culture form may be defined as the cell collection process.

The sixth culture form may be as follows: (a) the basal medium is supplied from the first supply unit 22a to the first circulation flow path 58; (b) the basal medium is not supplied from the second supply unit 22b to the second circulation flow path 62; and (c) a part of the culture medium is discarded from the first circulation flow path 58 to the waste liquid storage unit 26 via the first waste liquid flow path 76. That is, an operation of the cell culture apparatus 12 at the time of cell culture in the sixth culture form may be the same as the operation of the cell culture apparatus 12 at the time of cell culture in the second culture form (FIG. 13) except for (a).

In the sixth culture form, as in the second culture form, formula (2) to (5), formula (8), and formula (9) to (12) may be used. In the sixth culture form, similarly to the fifth culture form, the basal medium may be used instead of the complete medium. That is, formula (2) above becomes expression (2)′.

In the sixth culture form, the waste products and protein accumulated in the first region 44 may be discarded together with the culture medium into the waste liquid storage unit 26 via the first circulation flow path 58. According to the sixth culture form, the concentration of excessive protein in the first circulation flow path 58 may be reduced, and the waste products accumulated in the first region 44 may be discarded into the waste liquid storage unit 26.

The seventh culture form may be as follows: (a) the basal medium is supplied from the first supply unit 22a to the first circulation flow path 58; (b) the basal medium is supplied from the second supply unit 22b to the second circulation flow path 62; and (c) a part of the culture medium is discarded from the second circulation flow path 62 to the waste liquid storage unit 26 via the second waste liquid flow path 78. That is, an operation of the cell culture apparatus 12 at the time of cell culture in the seventh culture form may be the same as the operation of the cell culture apparatus 12 at the time of cell culture in the third culture form (FIG. 14) except for (a).

In the seventh culture form, as in the third culture form, formulas (1) to (6), formula (8), and formulas (9) and (13) may be used. In the seventh culture form, similarly to the fifth culture form, the basal medium may be used instead of the complete medium. That is, expression (2) above becomes expression (2)′.

According to the seventh culture form, as in the fifth culture form, the waste products accumulated in the first region 44 may be discarded into the waste liquid storage unit 26 without significantly changing the concentration of protein in the first circulation flow path 58.

The eighth culture form may be as follows: (a) the basal medium is supplied from the first supply unit 22a to the first circulation flow path 58; (b) the basal medium is supplied from the second supply unit 22b to the second circulation flow path 62; and (c) A part of the culture medium is discarded from the first circulation flow path 58 to the waste liquid storage unit 26 via the first waste liquid flow path 76. An operation of the cell culture apparatus 12 at the time of cell culture in the eighth culture form may be the same as the operation of the cell culture apparatus 12 at the time of cell culture in the fourth culture form (FIG. 15) except for (a).

In the eighth culture form, as in the fourth culture form, formula (2) to (5), formula (8) above, formula (9), formula (10), and formula (12) and (14) may be used. In the eighth culture form, similarly to the fifth culture form, the basal medium may be used instead of the complete medium. That is, expression (2) above becomes expression (2)′.

According to the eighth culture form, as in the sixth culture form, the concentration of excessive protein in the first circulation flow path 58 may be reduced, and the waste products accumulated in the first region 44 may be discarded into the waste liquid storage unit 26.

FIG. 16 is a flowchart illustrating a flow of the cell culture performed using the cell culture apparatus 12. A series of steps illustrated in FIG. 16 may be performed at step S5 illustrated in FIG. 10. The flow of the cell culture of adherent cells are described.

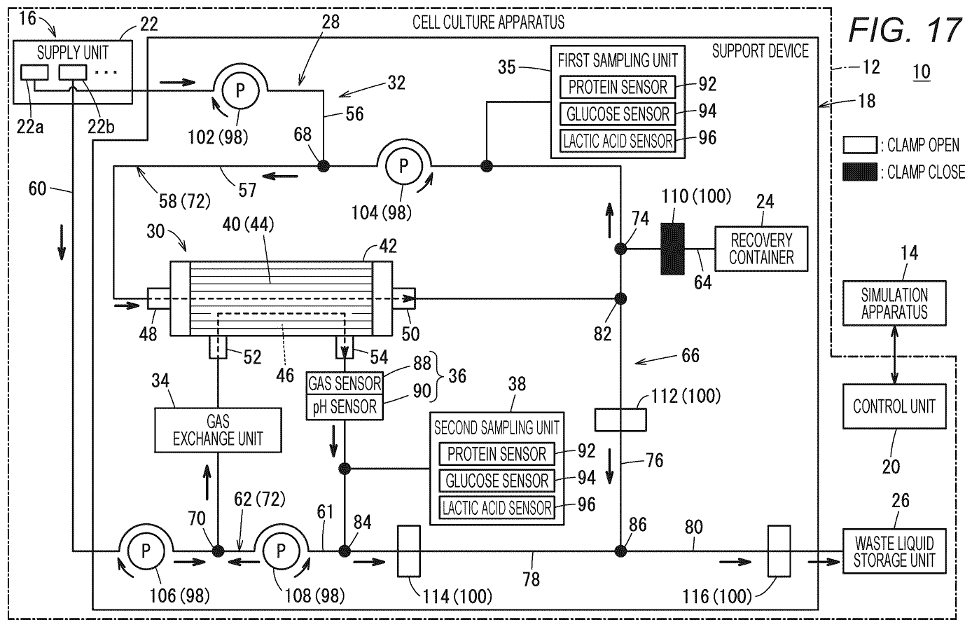

At step S11, the control unit 20 may be configured to perform seeding. As illustrated in FIG. 17, the pump control unit 122 may be configured to control each pump 98 and the clamp control unit 124 may be configured to control each clamp 100. The control unit 20 may be configured to control the first supply unit 22a to supply the cell fluid to the first supply flow path 56. Then, the cell fluid may be introduced from the first supply unit 22a to the first junction 68 of the first circulation flow path 58 via the first supply flow path 56. The cell fluid introduced into the first junction 68 may be guided from the first inlet port 48 to the first outlet port 50 through the first region 44. In the first region 44, the cells in the cell fluid may adhere to the inner surface of each hollow fiber membrane 40 of the bioreactor 30.

At step S12, the control unit 20 may be configured to start the cell culture in any one of the first to fourth culture forms. The control unit 20 may be configured to appropriately perform any one of the fifth to eighth culture forms. The control unit 20 may be configured to control the first supply unit 22a to supply the complete medium to the first supply flow path 56. Then, the complete medium may be introduced from the first supply unit 22a to the first junction 68 of the first circulation flow path 58 via the first supply flow path 56. The complete medium introduced into the first junction 68 may circulate in the first circulation flow path 58 including the first communication flow path 57, the first inlet port 48, the first region 44, and the first outlet port 50.

The control unit 20 may be configured to control the second supply unit 22b to supply the basal medium to the second supply flow path 60. Then, the basal medium may be introduced from the second supply unit 22b to the second junction 70 of the second circulation flow path 62 via the second supply flow path 60. The basal medium introduced into the second junction 70 may circulate in the second circulation flow path 62 including the second communication flow path 61, the second inlet port 52, the second region 46, and the second outlet port 54.

The gas exchange control unit 126 may be configured to control the gas exchange unit 34 to exchange the gas of the basal medium flowing through the second circulation flow path 62. That is, the gas exchange unit 34 may be configured to allow a gas of a predetermined or selected component to pass through the basal medium before flowing into the second inlet port 52. As a result, the gas concentration (e.g., oxygen gas concentration and carbon dioxide gas concentration) and the pH of the basal medium introduced into the second inlet port 52 of the bioreactor 30 may be adjusted to values suitable for the cell culture. In the bioreactor 30, the complete medium in the first region 44 and the basal medium in the second region 46 may be exchanged via the pores of each hollow fiber membrane 40. As a result, the gas concentration and the pH of the complete medium in the first region 44 may be adjusted.

The clamp control unit 124 may be configured to control the first waste liquid clamp 112 at an appropriate timing to open or close the first waste liquid flow path 76. When the first waste liquid flow path 76 is opened, a part of the complete medium in the first circulation flow path 58 may be guided to the third waste liquid flow path 80 via the first waste liquid flow path 76. The clamp control unit 124 may be configured to control the second waste liquid clamp 114 at an appropriate timing to open or close the second waste liquid flow path 78. When the second waste liquid flow path 78 is opened, a part of the basal medium in the second circulation flow path 62 may be guided to the third waste liquid flow path 80 via the second waste liquid flow path 78.

At step S13, the gas sensor 88 may be configured to measure the oxygen concentration of the culture medium and the carbon dioxide concentration of the culture medium. The pH sensor 90 may be configured to measures the pH of the culture medium. The gas sensor 88 and the pH sensor 90 may be configured to transmit measurement results to the control unit 20. The measurement unit 128 may be configured to acquire the measurement result from each sensor. The measurement unit 128 may be configured to store the acquired measurement result in the first storage unit 120. The gas sensor 88 and the pH sensor 90 may be configured to perform measurements until the cell culture ends.

At step S14, the control unit 20 may be configured to samples the culture medium. The pump control unit 122 and the gas exchange control unit 126 may be configured to control a pump (not illustrated) of the second sampling unit 38 and a clamp (not illustrated) of the second sampling unit 38 to sample the culture medium of the second circulation flow path 62. The sampled culture medium may flow to the waste liquid storage unit 26 via each sensor of the second sampling unit 38. Here, the first sampling unit 35 may be configured to sample the basal medium.

At step S15, the glucose sensor 94 may be configured to measure the glucose concentration of the culture medium and the lactic acid sensor 96 may be configured to measure the lactic acid concentration of the culture medium. The glucose sensor 94 and the lactic acid sensor 96 may be configured to transmit the measurement results to the control unit 20. The measurement unit 128 may be configured to acquire the measurement result from each sensor. The measurement unit 128 may be configured to store the acquired measurement result in the first storage unit 120. Here, the concentration of the basal medium sampled by the first sampling unit 35 may be measured.

At step S16, the control unit 20 may be configured to clean each sensor of the second sampling unit 38. The second sampling unit 38 may be provided with one or more pumps (not illustrated), one or more clamps (not illustrated), a cleaning liquid supply unit (not illustrated), the like, or any combination thereof. The pump control unit 122 may be configured to control the pump of the second sampling unit 38. The clamp control unit 124 may be configured to control the clamp of the second sampling unit 38. The control unit 20 may be configured to control the cleaning liquid supply unit. Then, the cleaning liquid may flow from the cleaning liquid supply unit to each sensor of the second sampling unit 38. As a result, each sensor of the second sampling unit 38 may be cleaned. The cleaning liquid that cleans each sensor of the second sampling unit 38 may then flow to the waste liquid storage unit 26.

At step S17, the control unit 20 may be configured to determine whether to end the cell culture on the basis of the measurement result measured by each sensor of the second sampling unit 38. In a case where the control unit 20 determines to end the cell culture (step S17: YES), the processing may proceed to step S18. In contrast, in a case where the control unit 20 determines to continue the cell culture (step S17: NO), the processing may return to step S14.

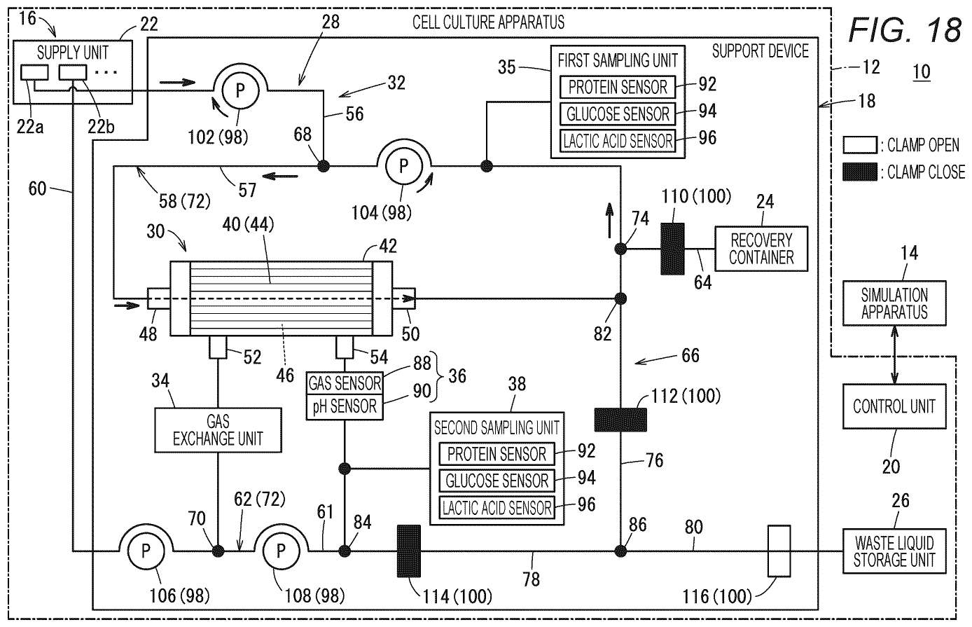

At step S18, the control unit 20 may be configured to performs cell detachment. As illustrated in FIG. 18, the pump control unit 122 may be configured to turn off the second supply pump 106 and the second circulation pump 108. As illustrated in FIG. 18, the clamp control unit 124 may be configured to control the first waste liquid clamp 112 and the second waste liquid clamp 114, for example, to close the first waste liquid flow path 76 and the second waste liquid flow path 78. The control unit 20 may be configured to control the supply unit 22 to supply the detaching liquid to the first supply flow path 56. Then, the detaching liquid may be guided from the supply unit 22 to the bioreactor 30 via the first supply flow path 56 and the first circulation flow path 58. In the bioreactor 30, the detaching liquid may detach the cultured cells from the inner surface of each hollow fiber membrane 40.

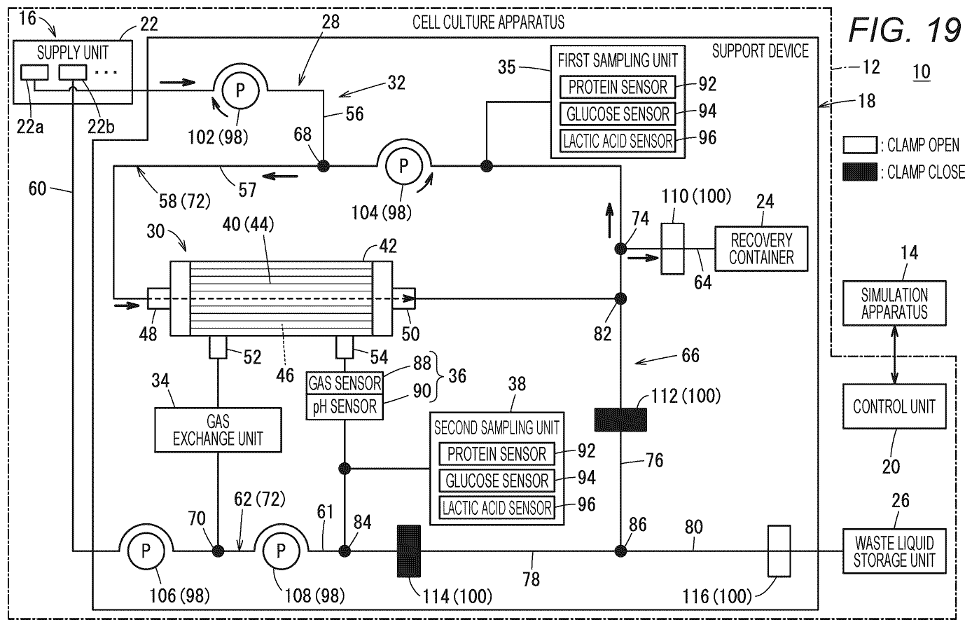

At step S19, the control unit 20 may be configured to recover the cells. As illustrated in FIG. 19, the clamp control unit 124 may be configured to control the recovery clamp 110 to open the recovery flow path 64. Then, the liquid containing the cells in the first circulation flow path 58 may be guided to the recovery container 24 via the recovery flow path 64. As a result, a series of steps of the cell culture may end.

As illustrated in FIG. 10, steps S1 to S6 may be repeatedly performed. For example, steps S1 to S6 may be performed N times (N≥2). At N-th step S1, the result measured in the step of the cell culture of (N−1)-th step S5 may be used as the proliferation data. In this case, the acquisition unit 140 of the simulation unit 132 may be configured to acquire data of the measurement result from the first storage unit 120 of the control unit 20. Each of the glucose measurement result and the lactic acid measurement result stored in the first storage unit 120 may be concentration data. Each of the oxygen measurement result and the carbon dioxide measurement result stored in the first storage unit 120 may be partial pressure data. In a case where the (N−1)-th measurement result is designated by the input unit 130, the acquisition unit 140 may be configured to convert the concentration data and the partial pressure data into metabolic speed data. The simulation execution unit 142 may be configured to simulate the cell culture using the converted data.

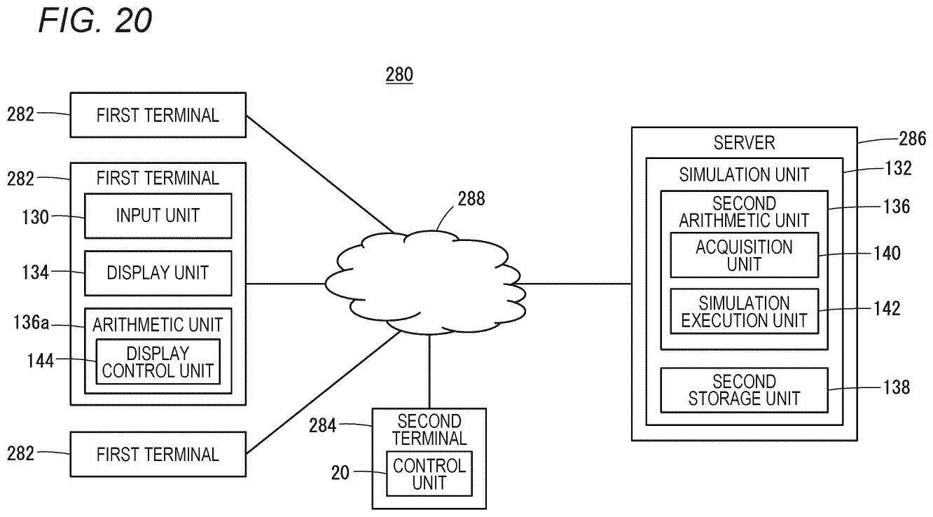

FIG. 20 is a diagram illustrating a configuration of a simulation system 280. The simulation system 280 illustrated in FIG. 20 may be used instead of the simulation apparatus 14 illustrated in FIG. 3. In FIG. 20, the components that are the same as those in FIG. 3 are denoted by the same reference numerals. The simulation system 280 may include at least one first terminal 282, at least one second terminal 284, and a server 286.

As the first terminal 282, a personal computer, a smartphone, a tablet, the like, or any combination thereof may be used. The first terminal 282 may include the input unit 130, an arithmetic unit 136a, and the display unit 134. The arithmetic unit 136a may include a processing circuit, such as a processor. The processor may be configured to function as the display control unit 144 by executing a program stored in a memory (not illustrated). The first terminal 282 may be connected to a communication network 288 via a communication device (not illustrated).

As the second terminal 284, a personal computer, a smartphone, a tablet, the like, or any combination thereof may be used. The second terminal 284 may include the control unit 20. The second terminal 284 may be connected to the communication network 288 via a communication device not illustrated.

The server 286 may include the simulation unit 132. The server 286 may be connected to the communication network 288 via a communication device (not illustrated). In at least one example embodiment, the server 286 may include a cloud server.

The communication network 288 may include a local area network (LAN) or a wide area network (WAN). The first terminal 282, the second terminal 284, and the server 286 may communicate with one another via the communication network 288.

When the user operates the input unit 130 to input data, the first terminal 282 may be configured to transmit each piece of data to the server 286. The simulation execution unit 142 of the server 286 may be configured to perform simulation using the data acquired from the first terminal 282. The server 286 may be configured to transmit the simulation result to the first terminal 282. The first terminal 282 may be configured to acquire the simulation result from the server 286. The display control unit 144 may be configured to allow the display unit 134 to display the simulation result. The second terminal 284 may be configured to acquire the data from the server 286.

The simulation may be executed using not only the supply speed (F_i_b) and the deterioration speeds (L_i_b1, L_i_b2) but also the exchange speeds (E_t_m1, E_t_m2) thereby improving. the accuracy of the simulation of the change in concentration of. The user can appropriately control (i.e., adjust) an amount of protein by reflecting the simulation result in the actual culture.

The simulation may be executed using the consumption speed (L_b_c) thereby further improving the accuracy of the simulation of the change in concentration of protein.

The simulation may be executed using the discard speeds (L_t_w1, L_t_w2) thereby further improving the accuracy of the simulation of the change in concentration of protein.

Certain features of the current technology are further illustrated in the following non-limiting examples.

A simulation of the change in concentration of protein was performed in accordance with step S2 of FIG. 10. The predicted value (i.e., calculated value) obtained by the simulation was compared with the measured value measured in the actual cell culture to evaluate the accuracy of the simulation. The concentration of protein did not sufficiently change in the culture process of the adherent cells but did significantly change in the culture process of the floating cells. The accuracy of the simulation was evaluated for the culture process of the floating cells. An error of the predicted value (i.e., calculated value) of the concentration of protein with respect to the measured value of the concentration of protein was within the allowable range (±5%×day or less). Accordingly, it was concluded that the simulation regarding the change in concentration of protein is appropriate.

The cell culture apparatus (e.g., cell culture apparatus 12) used in the examples were Quantum cell expansion systems (manufactured by Terumo BCT, Inc.) or Quantum Flex™ cell expansion systems (manufactured by Terumo BCT, Inc.). Hereinafter, the Quantum cell expansion system is referred to as a “first culture apparatus”, and the Quantum Flex™ cell expansion system is referred to as a “second culture apparatus”. A size of the bioreactor (e.g., bioreactor 30) is different between the first culture apparatus and the second culture apparatus. For example, the size of the bioreactor of the second culture apparatus may be 1/10 the size of the bioreactor of the first culture apparatus. At least one of the first culture apparatus and the second culture apparatus may change the size of the bioreactor. The hollow fiber membrane (e.g., hollow fiber membrane 40) provided in the culture apparatus may have a molecular weight cutoff of greater than or equal to about 5 kDa to less than or equal to about 20 kDa. The molecular weight cutoff of the hollow fiber membrane of the first culture apparatus and the second culture apparatus may be about 17 kDa.

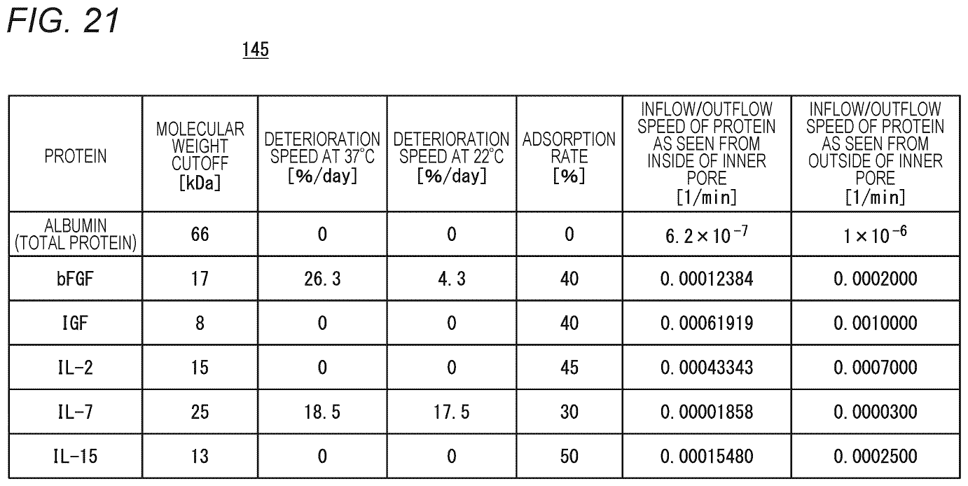

To perform the simulation, it is necessary to specify the parameter information (e.g., parameter information 145 illustrated in FIG. 4). The numerical values of the parameter information regarding several types of proteins were specified before performing the simulation. FIG. 21 illustrates the numerical values of the parameter information as specified. FIG. 21 is a table illustrating the example numerical values of the parameter information. The numerical values for the following proteins albumin (total protein), bFGF, IGF, IL-2, IL-7, and IL-15 have been specified in this example.

A culture medium containing protein of a predetermined concentration was placed in a temperature environment of 37° C. The concentration of protein was measured after a lapse of a predetermined or selected time. The numerical value of “the deterioration speed of protein at 37° C.” has been specified out of the parameter information on the basis of the predetermined concentration and the measured concentration. The numerical value of “the deterioration speed of protein at 22° C.” has been specified out of the parameter information by the method similar to the method of specifying “the deterioration speed of protein at 37° C.”.

The first culture apparatus was operated so that the flow path through which the culture medium flows was the same as that in the first culture form described above. At that time, a culture medium containing protein and free from cells was set in a first supply unit (e.g., first supply unit 22a). Supply and discard of the culture medium was executed according to the first culture form for a predetermined or selected period. Further, sampling was periodically performed in each of a first sampling unit (e.g., first sampling unit 35) and a second sampling unit (e.g., second sampling unit 38). The concentration of protein was measured in each culture medium. The numerical values of “inflow/outflow speed of protein as seen from inside of inner pore” and the numerical values of “inflow/outflow speed of protein as seen from outside of inner pore” were specified on the basis of the measurement result.

The concentration of protein (Pro_IC) was calculated in a first circulation flow path (e.g., first circulation flow path 58), for example, using formulas (1) to (5). At that time, the numerical value of “deterioration speed of protein at 37° C.” was substituted into Deg_37. The numerical value of “deterioration speed of protein at 22° C. was substituted into Deg_22. The numerical value of “inflow/outflow speed of protein as seen from inside of inner pore” was substituted into k_EtoI. In contrast, each of optional numerical values not smaller than 1 (for example, 0%, 10%, . . . , 90%, 100% and the like) were substituted into A_r corresponding to the adsorption rate out of the parameter information 145. Further, a concentration line (referred to as a predicted concentration line) was obtained that indicates the transition of the predicted value (i.e., calculated value) of the concentration of protein in a predetermined period. The predicted concentration line was obtained for each numerical value substituted into A_r. A concentration line (referred to as a measured concentration line) was also obtained that indicates the transition of the measured value of the concentration of protein in a predetermined period.

The predicted concentration line obtained for each A_r was compared with the measured concentration line. T The predicted concentration line closest to the measured concentration line was selected and specified A_r corresponding to the selected predicted concentration line as the “adsorption rate” included in the parameter information.

Each numerical value illustrated in FIG. 21 was specified by the methods. The numerical value of temperature of protein deterioration depends on a protein manufacturer. The numerical value of the adsorption rate depends on the form of the bioreactor of the culture apparatus. As described above, the numerical value of the parameter information changes due to various factors. That is, the numerical values illustrated in FIG. 21 are not unique values of each protein but reference values.

From the cell culture experiment and simulation to specify the parameters, the following was found:
Regarding albumin (total protein) larger than the molecular weight cutoff of the hollow fiber membrane, the predicted concentration line and the measured concentration line substantially coincided with each other.
Regarding protein not larger than the molecular weight cutoff of the hollow fiber membrane, the concentration tended to increase in both the first circulation flow path and the second circulation flow path, and the predicted concentration line and the measured concentration line substantially coincided with each other.
Regarding protein other than albumin (total protein), the predicted value tended to be higher than the measured value. Assuming that the protein adsorbs in the flow path, a ratio indicating the concentration similar to that of the measured value can be calculated and used as the correction rate (adsorption rate).
   As a result, “deterioration speed” and “exchange speed” (numerical values of “inflow/outflow speed of protein as seen from inside of inner pore” and “inflow/outflow speed of protein as seen from outside of inner pore”), and “correction rate (adsorption rate)” unique to each protein could be set.

After specifying the numerical values illustrated in FIG. 21, the accuracy of simulation for each of the cell culture was evaluated using the first culture apparatus and the cell culture using the second culture apparatus.

### Example 1

Three cell culture experiments under different culture conditions were conducted using the first culture apparatus to generate three specimens (specimen 1, specimen 2, and specimen 3) of the floating cells. In each of the three cell culture experiments, cell culture was performed for seven days, and the concentration of protein was periodically measured. Apart from this, a simulation regarding the change in concentration of protein was performed on the basis of the culture condition of each of the three specimens.

Execution conditions of the three cell culture experiments and the three simulations were as follows:
Jurkat cells were used as the floating cells to be cultured.
Complete medium containing total protein as protein was used.
The cell culture circuit 16 was primed with PBS, and after the priming, the bag filled with the complete medium was connected to the first supply flow path.
Gas conditioning was performed, and cells were seeded in the first circulation flow path.
In an initial stage of the cell culture period (seven days), a series of processes including the cell culture step, a cell dispersion step, and a cell collection step according to the first culture form were repeatedly performed. At the cell dispersion step, the supply and discard of the culture medium were stopped, and the culture medium was circulated in the first circulation flow path. At the cell collection step, the cells diffused into the first circulation flow path at the cell dispersion step were collected inside the bioreactor. Here, the cell collection step was performed according to the fifth culture form.
At the cell dispersion step, all clamps (e.g., recovery clamp 110, first waste liquid clamp 112, second waste liquid clamp 114, and third waste liquid clamp 116) are closed. In this state, the concentration of protein does not change. Therefore, there is no calculation expression of the protein concentration corresponding to the cell dispersion step.
In middle to later stages of the cell culture period (seven days), a series of processes including the cell culture step, a cell dispersion step, and a cell collection step according to the third culture form were repeatedly performed. Here, the cell collection step was performed according to the fifth culture form.

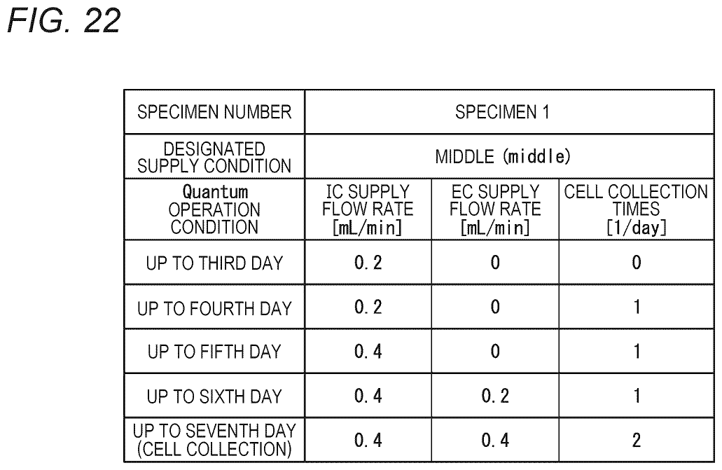

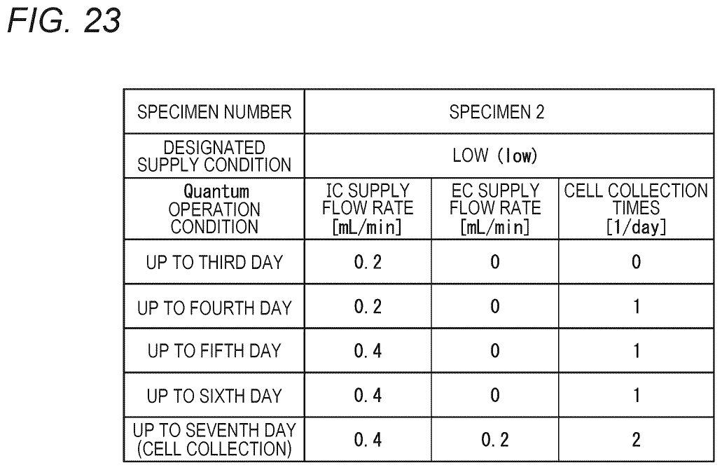

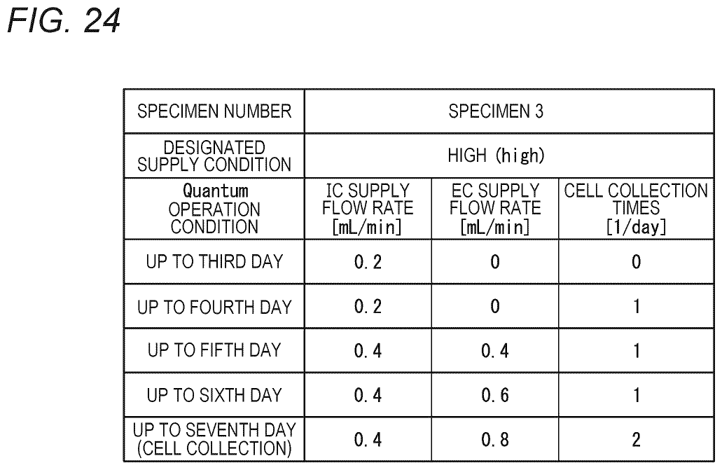

FIGS. 22 to 24 are tables illustrating the culture conditions. FIG. 22 illustrates the culture condition of the specimen 1. FIG. 23 illustrates the culture condition of the specimen 2. FIG. 24 illustrates the culture condition of the specimen 3. An “IC supply flow rate” illustrated in each table is the flow rate of the complete medium supplied to the first circulation flow path by a first supply pump (e.g., first supply pump 102). The “IC supply flow rate” corresponds to IC_ir of formula (2). An “EC supply flow rate” illustrated in each table is the flow rate of the basal medium supplied to the second circulation flow path by a second supply pump (e.g., second supply pump 106). The “EC supply flow rate” corresponds to EC_ir of formula (13).

As illustrated in FIGS. 22 to 24, in the three cell culture experiments, the flow rates of the complete medium and the basal medium and timings of switching from the first culture form to the third culture form are different from each other. In each table, a period in which the “EC supply flow rate” is zero means that the first culture form is being performed. In each Table, a period in which the “EC supply flow rate” is other than zero means that the third culture form is being performed.

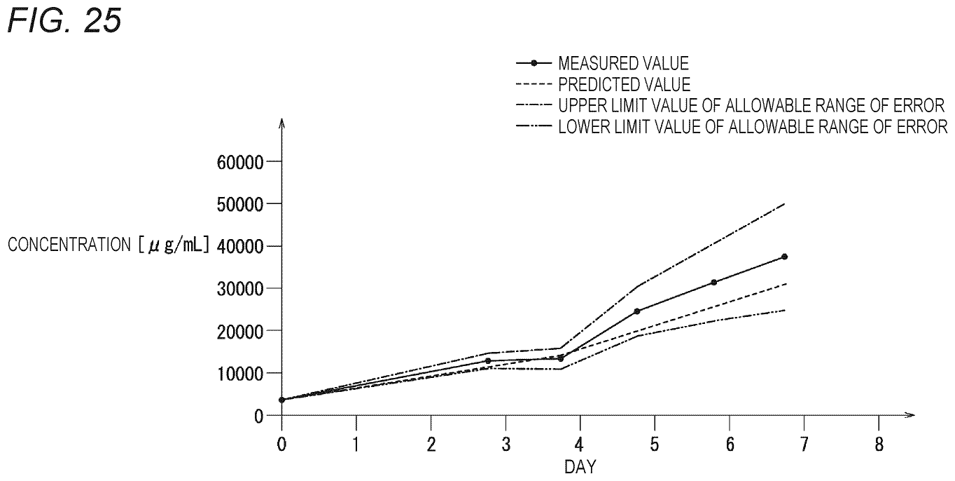

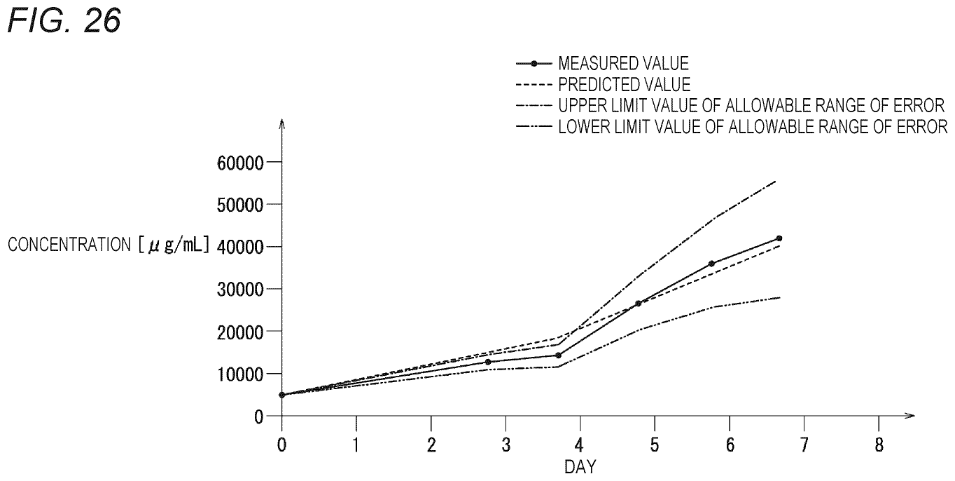

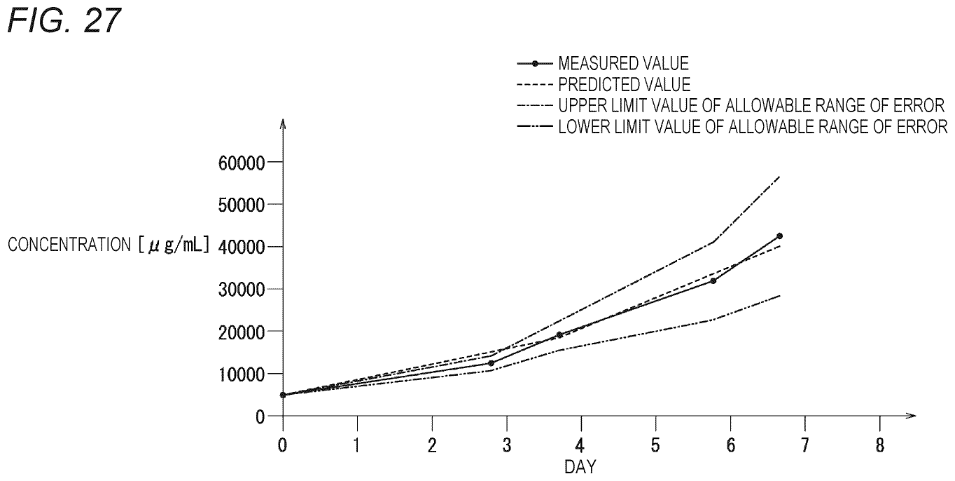

FIGS. 25 to 27 are graphical illustrations demonstrating the transition of the concentration of protein in the first circulation flow path. The graphical illustration in FIG. 25 demonstrates the transition of the concentration of protein in the experiment in which the specimen 1 was cultured, where the x-axis represents days and the y-axis represents concentration (μg/mL). The graphical illustration in FIG. 26 demonstrates the transition of the concentration of protein in the experiment in which the specimen 2 was cultured, where the x-axis represents days and the y-axis represents concentration (μg/mL). The graphical illustration in FIG. 27 demonstrates the transition of the concentration of protein in the experiment in which the specimen 3 was cultured, where the x-axis represents days and the y-axis represents concentration (μg/mL). In FIGS. 25-27, the solid line is the measured concentration line indicating the transition of the measured value of the protein (total protein) concentration; the broken line is a predicted concentration line indicating the transition of the predicted value (calculated value) of the protein (total protein) concentration; the dash-dot line is a line indicating an upper limit value of an allowable range of an error of the prediction value with respect to the measurement value; And the a two-dot chain line is a line indicating a lower limit value of the allowable range of the error of the prediction value with respect to the measurement value. The allowable range of the error is ±5%×day.

As illustrated in FIGS. 25-27, each predicted concentration line for the specimens 1 to 3 generally fell within the allowable range (between the lower limit value and the upper limit value) of the error. That is, the predicted concentration line regarding each of the specimens 1 to 3 fell within the range of the measured concentration line±5%×day in the middle to later stage of culture (after fourth day) in which cell proliferation was active. The predicted concentration line regarding each of the specimens 1 to 3 fell within the range of the measured concentration line±5%×day on a final day of culture.

### Example 2

A cell culture experiment using the second culture apparatus was conducted to generate a specimen of floating cells. In this example, cell culture was performed for seven days, and the concentration of protein was periodically measured. Otherwise, the simulation regarding the change in concentration of protein was performed on the basis of the culture condition of the specimen.

Execution conditions of the cell culture experiment and the simulation were as follows:
Jurkat cells were used as the floating cells to be cultured.
Complete medium containing total protein as protein was used.
Gas conditioning was performed, and cells were seeded in the first circulation flow path 58.
In an entire period of the cell culture period (seven days), a series of processes including the cell culture step, a cell dispersion step, and a cell collection step according to the first culture form was repeatedly performed. Here, the cell collection step was performed according to the first culture form.

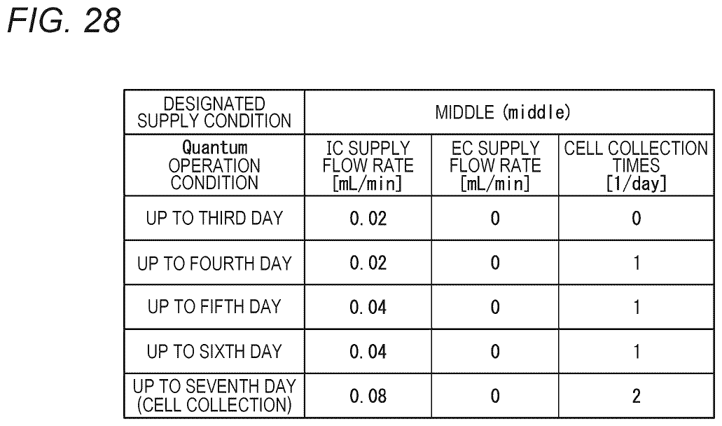

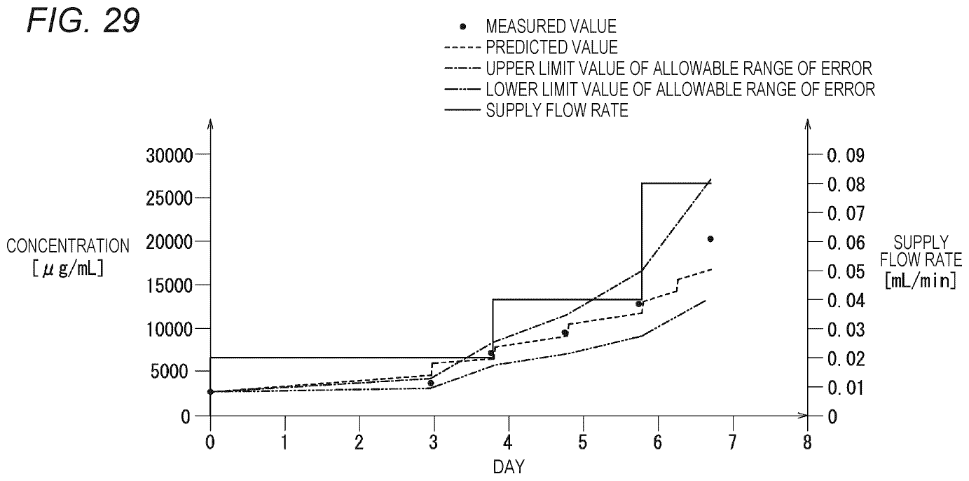

FIG. 28 is a table illustrating the culture condition. FIG. 29 is a graphical illustration demonstrating the transition of the concentration of protein in the first circulation flow path, where the x-axis represents days and the y-axis represents concentration (μg/mL). In FIG. 29, the broken line is a predicted concentration line indicating the transition of the predicted value (calculated value) of the protein (total protein) concentration; the dash-dot line is a line indicating an upper limit value of an allowable range of an error of the prediction value with respect to the measurement value; and the a two-dot chain line is a line indicating a lower limit value of the allowable range of the error of the prediction value with respect to the measurement value. The allowable range of the error was ±5%×day.

As illustrated in FIG. 29, the predicted concentration line regarding the specimen generally fell within the allowable range (between the lower limit value and the upper limit value) of the error. That is, the predicted concentration line regarding the specimen fell within the range of the measured concentration line±58×day in the middle to later (after third day) stages of culture in which cell proliferation was active. The predicted concentration line regarding the specimen fell within the range of the measured concentration line±5%×day on the final day of culture.

In examples 1 and 2, using the two culture apparatuses having different sizes, the error of the predicted value with respect to the measured value of the concentration of protein was within the allowable range. Accordingly, it was configured that the simulation regarding the change in concentration of protein is appropriate.

The simulation is not affected by the culture condition. The simulation of the concentration of protein can be performed without being affected by a combination of a plurality of culture forms, switching of a plurality of culture forms, a culture scale, the like, or any combination thereof.

### Example 3

In examples 1 and 2 described above, the measured value of the concentration of protein in the cell culture experiment of the floating cells (i.e., example) was compared with the predicted value (i.e., calculated value) of the concentration of protein in the simulation. In an example where the cell culture of the adherent cells is performed, the following applies:
HEK293 cells are used as the adherent cells to be cultured.
Complete medium containing total protein as protein is used.
After priming the cell culture circuit with PBS, the cell culture circuit is precoated with cellular adhesion factors (vitronectin and fibronectin), and after the precoating, the bag filled with the complete medium is connected to the first supply flow path.
Gas conditioning is performed, and cells are seeded in the first circulation flow path.
In the initial stage of the cell culture period, the cell culture step according to the second culture form is performed.
In the middle to later stages of the cell culture period, the cell culture step according to the fourth culture form is performed.

In the second culture form and the fourth culture form, which are culture forms of the adherent cells, the complete medium is supplied to the first circulation flow path, and the culture medium is discarded from the first circulation flow path to the waste liquid storage unit via a first waste liquid flow path (e.g., first waste liquid flow path 76). That is, the culture medium is not discarded from the second circulation flow path. Therefore, the concentration of protein in the first circulation flow path is substantially constant.

1. A simulation apparatus for simulating cell proliferation in a cell culture apparatus, the simulation apparatus comprising:
a simulation execution unit configured to simulate a change in concentration of a protein in a circulation path of the cell culture apparatus using a supply speed, an exchange speed, and a deterioration speed,
a predetermined or selected culture condition being applied in the circulation path,
the supply speed being a first speed at which a first concentration of the protein in the circulation path changes as a result of the supply of a culture medium to the circulation path,
the exchange speed being a second speed at which a second concentration of the protein in the circulation path changes as a result of inflow and outflow of the culture medium between the circulation path and an outer region of the circulation path, and
the deterioration speed being a third speed at which a third concentration of the protein in the circulation path changes as a result of deterioration of the protein.

2. The simulation apparatus of claim 1, further comprising:
a display control unit configured to control a display unit of the cell culture apparatus including to display information according to the concentration change obtained by the simulation.

3. The simulation apparatus of claim 1, wherein the simulation execution unit is further configured to use a consumption speed when simulating the change in the concentration of the protein in the circulation path, the consumption speed being a fourth speed at which a fourth concentration of the protein in the circulation path changes as a result of consumption of the protein by the cells.

4. The simulation apparatus of claim 1, wherein the simulation execution unit is further configured to use a discard speed when simulating the change in the concentration of the protein changes in the circulation path, the discard speed being a fifth speed at which a fifth concentration of the protein in the circulation path changes as a result of a part of the culture medium being discarded via the circulation path.

5. The simulation apparatus of claim 1, wherein the simulation execution unit is further configured to use an adsorption rate when simulating the change in concentration of the protein in the circulation path, the adsorption rate being a sixth concentration of protein in the circulation path that cannot be consumed by the cells because the sixth concentration of protein is adsorbed, aggregated, or deposited within or outside of the circulation path.

6. The simulation apparatus of claim 1, wherein the circulation path is an inner circulation path defined by an inner channel of a hollow fiber membrane of the cell culture apparatus and the cell culture apparatus is configured to supply a culture medium not containing the protein to an outer circulation path, the outer circulation path being defined by a region between the hollow fiber membrane and a housing that stores the hollow fiber membrane.

7. The simulation apparatus of claim 6, wherein the cells proliferated by the cell culture apparatus include adherent cells, and the cell culture apparatus is configured to discard the culture medium via a waste liquid flow path that is in communication with the inner circulation path.

8. The simulation apparatus of claim 6, wherein the cells proliferated by the cell culture apparatus include floating cells, and the cell culture apparatus is configured to discard the culture medium via a waste liquid flow path that is in communication with the outer circulation path.

9. The simulation apparatus of claim 1, wherein
the circulation path is an inner circulation path defined by an inner channel of a hollow fiber membrane of the cell culture apparatus and the cell culture apparatus further includes an outer circulation path defined between the hollow fiber membrane and a housing that stores the hollow fiber membrane, and
if a part of the culture medium is discarded via the outer circulation path during cell culture, the simulation execution unit is further configured to simulate a change in concentration of the protein in the outer circulation path using the exchange speed, the deterioration speed, and a discard speed,
the discard speed being a speed at which a concentration of the protein changes in the outer circulation as a result of a part of the culture medium being discarded via the outer circulation path.

10. A simulation system for simulating cell proliferation in a cell culture apparatus, the simulation system comprising:
a simulation execution unit configured to simulate a change in concentration of a protein in an inner circulation path of the cell culture apparatus using a supply speed, an exchange speed, and a deterioration speed,
a predetermined or selected culture condition being applied in the inner circulation path,
the cell culture apparatus including a hollow fiber membrane and the inner circulation path defined by an inner channel of the hollow fiber membrane,
the supply speed being a first speed at which a first concentration of the protein in the inner circulation path changes as a result of the supply of a culture medium to the inner circulation path,
the exchange speed being a second speed at which a second concentration of the protein in the inner circulation path changes as a result of inflow and outflow of the culture medium between the inner circulation path and an outer region of the inner circulation path, and
the deterioration speed being a third speed at which a third concentration of the protein in the inner circulation path changes as a result of deterioration of the protein; and
a display control unit configured to control a display unit of the cell culture apparatus including to display information according to the change in concentration obtained by the simulation.

11. The simulation system of claim 10, wherein
the simulation execution unit is further configured to use at least one of a consumption speed, a discard speed, and an adsorption rate when simulating the change in the concentration of the protein in the circulation path,
the consumption speed being a fourth speed at which a fourth concentration of the protein in the circulation path changes as a result of consumption of the protein by the cells,
the discard speed being a fifth speed at which a fifth concentration of the protein in the circulation path changes as a result of a part of the culture medium being discarded via the circulation path, and
the adsorption rate being a sixth concentration of protein in the circulation path that cannot be consumed by the cells because the sixth concentration of protein is adsorbed, aggregated, or deposited within or outside of the circulation path.

12. A simulation method for simulating cell proliferation in a cell culture apparatus, the simulation method comprising:
simulating a change in concentration of a protein in a circulation path of the cell culture apparatus using a supply speed, an exchange speed, and a deterioration speed,
a predetermined or selected culture condition being applied in the circulation path,
the supply speed being a first speed at which a first concentration of the protein in the circulation path changes as a result of the supply of a culture medium to the circulation path,
the exchange speed being a second speed at which a second concentration of the protein in the circulation path changes as a result of inflow and outflow of the culture medium between the circulation path and an outer region of the circulation path, and
the deterioration speed being a third speed at which a third concentration of the protein in the circulation path changes as a result of deterioration of the protein.

13. The simulation method of claim 12, further comprising:
displaying information according to the change in concentration obtained by the simulation on a display unit of the cell culture apparatus.

14. The simulation method of claim 12, wherein the simulating of the change in the concentration of the protein in the circulation path of the cell culture apparatus further uses a consumption speed, the consumption speed being a fourth speed at which a fourth concentration of the protein in the circulation path changes as a result of consumption of the protein by the cells.

15. The simulation method of claim 12, wherein the simulating of the change in the concentration of the protein in the circulation path of the cell culture apparatus further uses a discard speed, the discard speed being a fifth speed at which a fifth concentration of the protein in the circulation path changes a result of a part of the culture medium being discarded via the circulation path.

16. The simulation method of claim 12, wherein the simulation of the change in the concentration of the protein in the circulation path of the cell culture apparatus further uses an adsorption rate, the adsorption rate being a sixth concentration of protein in the circulation path that cannot be consumed by the cells because the sixth concentration of protein is adsorbed, aggregated, or deposited within or outside of the circulation path.

17. The simulation method of claim 12, wherein
the circulation path is an inner circulation path defined by an inner channel of a hollow fiber membrane of the cell culture apparatus, and
the method further includes supplying a culture medium not containing the protein to an outer circulation path, the outer circulation path being defined by a region between the hollow fiber membrane and a housing that stores the hollow fiber membrane.

18. The simulation method of claim 16, wherein the cells proliferated by the cell culture apparatus include adherent cells, and the cell culture apparatus is configured to discard the culture medium via a waste liquid flow path that is in communication with the circulation path.

19. The simulation method of claim 16, wherein the cells proliferated by the cell culture apparatus include floating cells, and the cell culture apparatus is configured to discard the culture medium via a waste liquid flow path that is in communication with the outer circulation path.

20. The simulation method of claim 12, wherein
the circulation path is an inner circulation path defined by an inner channel of a hollow fiber membrane of the cell culture apparatus and the cell culture apparatus further includes an outer circulation path defined between the hollow fiber membrane and a housing that stores the hollow fiber membrane,
if a part of the culture medium is discarded via the outer circulation path during cell culture, the simulating of the change in the concentration of the protein in the circulation path of the cell culture apparatus further includes using a discard speed,
the discard speed being a speed a concentration of the protein changes in the outer circulation as a result of a part of the culture medium being discarded via the outer circulation path.
